(19) United States
(12) Patent Application Publication
(10) Pub. No.: US 2021/0155941 A1
(43) Pub. Date: May 27, 2021

(54) COMPOSITIONS AND METHODS FOR MAKING ENGINEERED T CELLS

(71) Applicant: KITE PHARMA EU B.V., Amsterdam (NL)

(72) Inventors: Gavin Matthew BENDLE, Amsterdam (NL); Carsten LINNEMANN, Amsterdam (NL); Deborah SCHRIKKEMA, Amsterdam (NL); Bianca WEISSBRICH, Amsterdam (NL)

(73) Assignee: KITE PHARMA EU B.V., Amsterdam (NL)

(21) Appl. No.: 17/253,467

(22) PCT Filed: Jun. 21, 2019
(86) PCT No.: PCT/IB2019/000658

§ 371 Date: Dec. 17, 2020

Related U.S. Application Data
(60) Provisional application No. 62/688,941, filed on Jun. 22, 2018.
(60) Provisional application No. 62/690,142, filed on Jun. 26, 2018.

(51) Int. Cl. C12N 15/64; C07K 14/725

(52) U.S. Cl. CPC C12N 15/64; C12N 2330/51; C12N 2310/141; C07K 14/7051

(57) ABSTRACT

The disclosure provides a vector which can be used in a method of generating engineered T cells for use in an autologous or allogeneic setting for engineered immunotherapy. The knockdown of endogenous TCR expression through a vector comprising a miRNA cassette allows for engineering of cells that efficiently express a therapeutic TCR.

### CROSS-REFERENCE TO RELATED APPLICATIONS

This application claims priority to U.S. Provisional Application No. 62/688,941, filed Jun. 22, 2018 and to U.S. Provisional Application No. 62/690,142, filed Jun. 26, 2018, both of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Jun. 21, 2019, is named KPI-024WO_SL.txt and is 20,580 bytes in size.

### BACKGROUND

Human cancers are by their nature comprised of normal cells that have undergone a genetic or epigenetic conversion to become abnormal cancer cells. In doing so, cancer cells begin to express proteins and other antigens that are distinct from those expressed by normal cells. These aberrant tumor antigens can be used by the body's immune system to specifically target and kill cancer cells. However, cancer cells employ various mechanisms to prevent immune cells, such as T and B lymphocytes, from successfully targeting cancer cells.

Current T cell therapies rely on enriched or modified human T cells to target and kill cancer cells in a patient. To increase the ability of T cells to target and kill a particular cancer cell, methods have been developed to engineer T cells to express constructs, which direct T cells to a particular target cancer cell. Engineered T cell receptors (TCRs), which comprise binding domains capable of interacting with a particular tumor antigen, allow T cells to target and kill cancer cells that express the particular tumor antigen.

A need exists for improved methods of generating TCRs and antigen receptor modified T cells for specifically targeting and killing cancer cells.

### SUMMARY

The present disclosure addresses this need by, among other things, providing compositions and methods comprising genetically engineered T cells. In particular, the present disclosure provides a retroviral vector encoding the amino acid sequences of human T cell receptor (TCR) α and β chains and a micro RNA (miRNA) cassette specific to the endogenous host TCR. In some embodiments, the retroviral vector encoding the amino acid sequences of human T cell receptor (TCR) α and β chains with variable domains are specific to a HLA-A*02:01/YMLDLQPET peptide-MHC (pMHC) complex. The nonamer peptide YMLDLQPET (SEQ ID NO: 1) is encoded by the Human Papilloma virus serotype 16 (HPV16) E7 protein (Amino acids 11-19) which is expressed in various HPV16-associated tumor cells. Hence, the TCR vector may be used to generate TCR transduced (td) T cells for cancer therapy. To ensure high level cell surface expression of the human TCR encoded within the TCR vector on TCR td T cells, the vector also contains a multiplexed miRNA cassette that targets the endogenous TCR chains in TCR td T cells for knockdown (miRαβ cassette). The miRαβ cassette contains TCR α and β specific miRNA (miR155_TRAC and AmiR_TRBC, respectively) that target the invariant constant domain of the TCR α and β chains respectively. The human TCR α and β chains encoded with the TCR vector are not targeted by these miRNA, as the DNA sequences encoding them are codon-optimized.

TCRs are proteins that allow T cells to identify cancer targets presented on the surface of cancer cells or inside cancer cells. Endogenous TCRs that are specific to a cancer can be isolated and then engineered into a large number of T cells that recognize and attack various types of solid and hematologic cancers.

In one aspect, the present disclosure provides a vector comprising a nucleic acid sequence encoding a recombinant therapeutic T cell receptor (TCR) specific to a Human Papilloma virus serotype 16 (HPV16) E7 protein peptide-MHC (pMHC) complex and a microRNA (miRNA) cassette targeting the constant domain of the endogenous human TCR α and β chains wherein the recombinant therapeutic TCR comprises a fully human constant region.

In some embodiments, the miRNA cassette comprises a nucleic acid sequence that is at least 90%, 91%, 92%, 93%, 94%, 95%, 96%, 97%, 98%, 99%, or 100% identical to any one of SEQ ID NO: 7-10.

In some embodiments, the miRNA cassette comprises a nucleic acid sequence that is at least 90%, 91%, 92%, 93%, 94%, 95%, 96%, 97%, 98%, 99%, or 100% identical any one of SEQ ID NO: 11-14.

In some embodiments, the miRNA cassette comprises a nucleic acid sequence that is at least 90%, 91%, 92%, 93%, 94%, 95%, 96%, 97%, 98%, 99%, or 100% identical to SEQ ID NO: 6.

In some embodiments, the vector is an adenoviral vector, an adenovirus-associated vector, a DNA vector, a lentiviral vector, a plasmid, a retroviral vector, or an RNA vector. In some embodiments, the vector is a viral vector. In some embodiments, the vector is a retroviral vector. In some embodiments, is the pMP71 retroviral vector.

In some embodiments, the vector is a lentiviral vector.

In some embodiments, the recombinant therapeutic TCR is specific to a peptide-MHC (pMHC) complex comprising the amino acid sequence of SEQ ID NO: 1.

In some embodiments, the recombinant therapeutic TCR is specific to an HLA-A*02:01/YMLDLQPET peptide-MHC (pMHC) complex.

In some embodiments, the recombinant therapeutic TCR comprises a TCR β chain, a 2A family member sequence and a TCR α chain. In some embodiments, the recombinant therapeutic TCR is in the configuration: TCR β chain-Furin cleavage site-Linker-P2A-TCR α chain. In some embodiments, the Furin cleavage site comprises the amino acid sequence RAKR (SEQ ID NO: 20). In some embodiments, the short linker comprises the amino acid sequence SGSG (SEQ ID NO: 18).

In some embodiments, the recombinant therapeutic TCR comprises an α chain that comprises the amino acid sequence of SEQ ID NO: 4. In some embodiments, the recombinant therapeutic TCR comprises an α chain that comprises the amino acid sequence of SEQ ID NO: 2. In some embodiments, the recombinant therapeutic TCR comprises an α chain that comprises an amino acid sequence that is at least 90%, 91%, 92%, 93%, 94%, 95%, 96%, 97%, 98%, or 99% identical to the amino acid sequence of SEQ ID NO: 4 or SEQ ID NO: 2. In some embodiments, the recombinant therapeutic TCR comprises an α chain that comprises a signal peptide according to SEQ ID NO: 23. In some embodiments, the recombinant therapeutic TCR comprises an α chain comprises a signal peptide according to SEQ ID NO: 22.

In some embodiments, the recombinant therapeutic TCR comprises a β chain that comprises the amino acid sequence of SEQ ID NO: 5. In some embodiments, the recombinant therapeutic TCR comprises a β chain that comprises the amino acid sequence of SEQ ID NO: 3. In some embodiments, the recombinant therapeutic TCR comprises an β chain that comprises an amino acid sequence that is at least 90%, 91%, 92%, 93%, 94%, 95%, 96%, 97%, 98%, or 99% identical to the amino acid sequence of SEQ ID NO: 5 or SEQ ID NO: 3.

In some embodiments, the recombinant therapeutic TCR α and TCR β chain is codon optimized.

In some embodiments, the recombinant therapeutic TCR comprises a fully human constant region.

In some embodiments, the miRNA cassette is present in the pMP71 retroviral vector between splice donor and splice acceptor sites downstream of the 5′LTR.

In one aspect, the present disclosure provides a vector substantially as described in FIG. 7.

In one aspect, the present disclosure provides a cell comprising the vector of described herein.

In some embodiments, the cell is an isolated hematopoietic stem cell, an embryonic stem cell, or an induced pluripotent stem cell.

In some embodiments, the cell is an induced pluripotent stem cell (iPSC) derived from a T cell or non-T cell.

In some embodiments, the cell is a T cell.

In some embodiments, the T cell is an allogeneic T cell, an autologous T cell, an engineered autologous T cell (eACT), or a tumor-infiltrating lymphocyte (TIL).

In some embodiments, the T cell is a CD4+ T cell. In some embodiments, the T cell is a CD8+ T cell. In some embodiments, the cell is an isolated cell.

In some embodiments, the T cell is an autologous T cell.

In some embodiments, the cell produces at least Interferon gamma (IFNγ) upon binding to pMHC.

In one aspect, the present disclosure provides a composition comprising a plurality of cells described herein.

In some embodiments, the composition comprises CD4+ or CD8+ cells. In some embodiments, the composition comprises CD4+ and CD8+ cells. In some embodiments, each cell in the plurality of cells is an autologous T cell. In some embodiments, the composition comprises at least one pharmaceutically acceptable excipient.

In one aspect, the present disclosure provides a composition comprising a vector described herein.

In one aspect, the present disclosure provides a method for manufacturing a cell expressing a therapeutic T cell receptor (TCR), comprising a step of transducing a cell with a vector described herein. In some embodiments, the cell is a lymphocyte isolated from a patient in need of treatment.

In some embodiments, the lymphocyte is a natural killer cell, a T cell, or a B cell. In some embodiments, the cell is an isolated hematopoietic stem cell, an embryonic stem cell, or an induced pluripotent stem cell.

In some embodiments, the cell is an induced pluripotent stem cell (iPSC) derived from a T cell or non-T cell.

In some embodiments, the method further comprises a step of differentiating the stem cell into T cells.

In some embodiments, the method further comprises a step of culturing the cell under conditions that promote cellular proliferation and/or T cell activation.

In some embodiments, the method further comprises a step of isolating desired T cells expressing a therapeutic T cell receptor (TCR). In some embodiments, the step of isolating desired T cells occurs after about six days of culturing. In some embodiments, the step of isolating desired T cells occurs after about seven, eight or nine days of culturing. In some embodiments, the step of isolating desired T cells occurs between about 6-10 days of culturing.

In some embodiments, the desired T cells express CD4+ and/or CD8+.

In one aspect, the present disclosure provides a method for treating a HPV associated cancer comprising administering to a subject in need thereof a cell or a composition described herein.

In some embodiments, the HPV associated cancer is HPV16-associated cancer.

In some embodiments, the HPV associated cancer is an oropharyngeal cancer or a cervical cancer.

Any aspect or embodiment described herein may be combined with any other aspect or embodiment as disclosed herein. While the present invention has been described in conjunction with the detailed description thereof, the foregoing description is intended to illustrate and not limit the scope of the present invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, dictionaries, documents, manuscripts, genomic database sequences, and scientific literature cited herein are hereby incorporated by reference.

Other features and advantages of the invention will be apparent from the Drawings and the following Detailed Description, including the Examples, and the claims.

### BRIEF DESCRIPTION OF THE DRAWINGS

The above and further features will be more clearly appreciated from the following detailed description when taken in conjunction with the accompanying drawings. The drawings however are for illustration purposes only, not for limitation.

FIGS. 1A and 1B show transduction efficiency (T_{deff}) of the TCR vector with the miRNAαβ cassette (FIG. 1B) and the vector without the miRNAαβ cassette (FIG. 1A) measured by flow cytometry using monoclonal antibodies (mAbs) specific for the TCR V gene elements used in the TCR (Vb5.2 mAb). Incorporation of a miRαβ cassette in the pMP71 A*02:01/HPV16 E7₁₁₋₁₉ fHC TCR vector leads to high level expression of the A*02:01/HPV16 E7₁₁₋₁₉ human TCR on transduced T cells. Data shown is representative for 9 donors in 3 different experiments.

FIGS. 2A and 2B show the percentage of cells expressing endogenous TCR TCRβ following transduction using the vector with the miRNAαβ cassette (FIG. 2B) and the vector without the miRNAαβ cassette (FIG. 2A) measured by flow cytometry. Incorporation of a miRαβ cassette in the pMP71-A*02:01/HPV16 E7₁₁₋₁₉ fHC TCR vector reduces expression of TCRαβ heterodimers containing endogenous TCRβ chains on TCR td T cells. Data shown is representative for 9 donors in 3 different experiments.

FIGS. 3A and 3B show intracellular interferon-γ (IFN-γ) levels measured by flow cytometry. Incorporation of a miRαβ cassette in the pMP71-A*02:01/HPV16 E7₁₁₋₁₉ fHC TCR vector enhances sensitivity and responsiveness of CD8+ TCR td T cells (FIG. 3A) and CD4+ TCR Td cells (FIG. 3B). Data shown is representative for 9 donors in 3 different experiments.

FIGS. 4A and 4B show intracellular interferon-γ (IFN-γ) levels measured by flow cytometry. Incorporation of a miRαβ cassette in the pMP71-A*02:01/HPV16 E71-19 fHC TCR vector enhances in vitro tumor reactivity of CD8+(FIG. 4A) and CD4+(FIG. 4B) TCR td T cells.

FIGS. 5A-5E show the A*02:01/HPV16 E7₁₁₋₁₉ TCR (TCR1) performs equally well in the pMP71-miRαβ-A*02:01/HPV16 E7₁₁₋₁₉ fHC TCR (human constant) vector compared to the pMSGV-fMC-Cys-LVLα (murine constant) clinical TCR vector in in vitro studies. The miRαβ TCR vector (SEQ ID NO: 15) reduced the expression of TCR α heterodimers containing endogenous TCR β chains on TCR td T cells (FIG. 5B). The miRαβ TCR vector (SEQ ID NO: 15) performed at least as well as the clinical TCR vector with respect to expression of the A*02:01/HPV16 E7₁₁₋₁₉-specific TCR on td T cells (FIG. 5A); antigen sensitivity of CD8+ and CD4+ TCR td T cells (FIG. 5C); tumor-reactivity as measured by IFN-γ production in CD8+ and CD4+ TCR td T cells (FIG. 5D) and cytotoxicity of TCR td T cells (FIG. 5E).

FIGS. 6A and 6B show change in tumor size (mm³) in NSG mice after injection with HPV16+/HLLA-A*02:01 human Caski tumor cells. Four days after tumor injection, human T cells transduced with the different TCR vector cassettes were transferred. The clinical TCR vector A*02:01/HPV16 E7₁₁₋₁₉ TCR performs equally well in the pMP71-miRαβ-A*02:01/HPV16 E7₁₁₋₁₉ fHC TCR vector compared to the clinical TCR vector (without a miRNAαβ cassette) in in vivo mouse studies. This revealed that the T cells transduced with either TCR vector showed an equivalent ability to control tumor outgrowth (FIG. 6A). Survival percentage at day 58 is shown in FIG. 6B.

FIG. 7 shows the vector map of the pMP71-miRαβ-A*02:01/HPV16 E7₁₁₋₁₉ fHC TCR vector.

### DEFINITIONS

In order for the present invention to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and.”

The term “and/or” where used herein is to be taken as specific disclosure of each of the two specified features or components with or without the other. Thus, the term “and/or” as used in a phrase such as “A and/or B” herein is intended to include A and B; A or B; A (alone), and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 1920, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., 10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2^{nd} ed., (2001), CRC Press; “The Dictionary of Cell & Molecular Biology”, 5^{th} ed., (2013), Academic Press; and “The Oxford Dictionary Of Biochemistry And Molecular Biology”, Cammack et al. eds., 2^{nd} ed., (2006), Oxford University Press, provide those of skill in the art with a general dictionary for many of the terms used in this disclosure.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intra-arterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal, or mucosal route of administration, for example, intranasally, vaginally, rectally, sublingually or topically. Administering can also be performed, for example, once, a plurality of times, and/or over one or more extended periods.

The term “antibody” (Ab) includes, without limitation, a glycoprotein immunoglobulin, which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2, and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

Antibodies can include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above. In certain embodiments, antibodies described herein refer to polyclonal antibody populations.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “antigen binding molecule,” “antigen binding portion,” or “antibody fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′)₂, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments, the antigen binding molecule binds to BCMA, CLL-1, or FLT3. In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv). In some embodiments, the antigen binding molecule comprises or consists of avimers.

As used herein, the term “variable region” or “variable domain” is used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

A T cell receptor (TCR) comprises a variable region, comprising V(D)J variable region segments, and this variable region confers upon the T cell its antigen binding specificity. As used herein, a TCR variable region or TCR variable domain (e.g., TCRα variable region or TCRβ variable region), comprises the region that includes TCR variable region segments (V(D)J region). Each of the two polypeptides that make up the TCR contains an extracellular domain comprising constant and variable regions, a transmembrane domain, and a cytoplasmic tail (the transmembrane domain and the cytoplasmic tail also being a part of the constant region). The variable region of the TCR determines its antigen specificity, and similar to immunoglobulins, comprises three hypervariable loop structures referred to as complementary determining regions (CDRs). In TCR chains, the first (CDR1) and second (CDR2) CDR loops typically contact the relatively less variable MHC component of the MHC:antigen complex. In contrast, the third CDR loop (CDR3), which is largely responsible for making the contact with the presented antigen, is the most highly variable.

As used herein, an antigen binding molecule, an antibody, or an antigen binding molecule thereof “cross-competes” with a reference antibody or an antigen binding molecule thereof if the interaction between an antigen and the first binding molecule, an antibody, or an antigen binding molecule thereof blocks, limits, inhibits, or otherwise reduces the ability of the reference binding molecule, reference antibody, or an antigen binding molecule thereof to interact with the antigen. Cross competition can be complete, e.g., binding of the binding molecule to the antigen completely blocks the ability of the reference binding molecule to bind the antigen, or it can be partial, e.g., binding of the binding molecule to the antigen reduces the ability of the reference binding molecule to bind the antigen. In certain embodiments, an antigen binding molecule that cross-competes with a reference antigen binding molecule binds the same or an overlapping epitope as the reference antigen binding molecule. In other embodiments, the antigen binding molecule that cross-competes with a reference antigen binding molecule binds a different epitope as the reference antigen binding molecule. Numerous types of competitive binding assays can be used to determine if one antigen binding molecule competes with another, for example: solid phase direct or indirect radioimmunoassay (RIA); solid phase direct or indirect enzyme immunoassay (EIA); sandwich competition assay (Stahli et al., 1983, Methods in Enzymology 9:242-253); solid phase direct biotin-avidin EIA (Kirkland et al., 1986, J. Immunol. 137:3614-3619); solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15), solid phase direct biotin-avidin EIA (Cheung, et al., 1990, Virology 176:546-552), and direct labeled RIA (Moldenhauer et al., 1990, Scand. J. Immunol. 32:77-82).

An “antigen” refers to any molecule that provokes an immune response or is capable of being bound by an antibody or an antigen binding molecule (e.g., a TCR). The immune response may involve either antibody production, or the activation of specific immunologically-competent cells, or both. A person of skill in the art would readily understand that any macromolecule, including virtually all proteins or peptides, could serve as an antigen. An antigen can be endogenously expressed, i.e. expressed by genomic DNA, or can be recombinantly expressed. An antigen can be specific to a certain tissue, such as a cancer cell, or it can be broadly expressed. In addition, fragments of larger molecules can act as antigens. In one embodiment, antigens are tumor antigens. In some embodiments, the antigen is a tumor antigen peptide:MHC complex.

The term “allogeneic” refers to any material derived from one individual, which is then introduced to another individual of the same species, e.g., allogeneic T cell transplantation.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

A “cancer” refers to a broad group of various diseases characterized by the uncontrolled growth of abnormal cells in the body. Unregulated cell division and growth results in the formation of malignant tumors that invade neighboring cells or tissues and may metastasize to distant parts of the body through the lymphatic system or bloodstream. As used herein the terms “cancer” or “cancer tissue” include both solid and liquid tumors. Examples of cancers that can be treated by the methods of the present invention include, but are not limited to, cancers of the immune system including lymphoma, leukemia, myeloma, and other leukocyte malignancies. In some embodiments, the methods of the present invention can be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers. In one particular embodiment, the cancer is an HPV related cancer, e.g., cervical or head and neck cancer. The particular cancer can be responsive to chemo- or radiation therapy or the cancer can be refractory. A refractor cancer refers to a cancer that is not amendable to surgical intervention and the cancer is either initially unresponsive to chemo- or radiation therapy or the cancer becomes unresponsive over time. The TCR disclosed herein can be particularly useful in the treatment of patients having solid tumors.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

A “cytokine,” as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) 7. Examples of pro-inflammatory cytokines include, but are not limited to, IL-1a, IL-1b, IL-6, IL-13, IL-17a, tumor necrosis factor (TNF)-α, TNF-β, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), γ-induced protein 10 (IP-10), and thymus and activation regulated chemokine (TARC or CCL17).

A “therapeutically effective amount,” “effective dose,” “effective amount,” or “therapeutically effective dosage” of a therapeutic agent, e.g., engineered TCR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T-cells play a major role in cell-mediated-immunity (no antibody involvement). Its T-cell receptors (TCR) differentiate themselves from other lymphocyte types. The thymus, a specialized organ of the immune system, is primarily responsible for the T cell's maturation. There are six types of T-cells, namely: Helper T-cells (e.g., CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory TSCM cells, like naive cells, are CD45RO−, CCR7+, CD45RA+, CD62L+(L-selectin), CD27+, CD28+ and IL-7Ra+, but they also express large amounts of CD95, IL-2RP, CXCR3, and LFA-1, and show numerous functional attributes distinctive of memory cells); (ii) central memory TCM cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T-cells (Tregs, suppressor T cells, or CD4+CD25+ regulatory T cells), Natural Killer T-cells (NKT) and F Delta T-cells. B-cells, on the other hand, play a principal role in humoral immunity (with antibody involvement). They make antibodies and antigens, perform the role of antigen-presenting cells (APCs), and turn into memory B-cells after activation by antigen interaction. In mammals, immature B-cells are formed in the bone marrow.

The term “genetically engineered”, “engineered”, or “modified” refers to a method of modifying a cell, including, but not limited to, creating a deficiency in a gene by deleting a coding or non-coding region or a portion thereof or by antisense technology, or increasing expression of a protein introducing a coding region or a portion thereof. In some embodiments, the cell that is modified is a stem cell (e.g., hematopoietic stem cell (HSC), embryonic stem cell (ES), induced pluripotent stem (iPS) cell), lymphocyte (e.g., a T cell), which can be obtained either from a patient or a donor. The cell can be modified to express an exogenous construct, such as, e.g., a pre-TCR α protein or a T cell receptor (TCR), which may be incorporated into the cell's genome.

MicroRNAs (miRNAs) are short non-coding RNA oligonucleotides that can be used for gene regulation (e.g., knockdown, gene silencing). As used herein, when delivered to a target cell, a miRNA expression cassette reduces expression of the target gene (e.g., endogenous TCRα and/or TCRβ).

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing, or otherwise modifying an immune response. Examples of immunotherapy include, but are not limited to, T cell therapies. T cell therapy can include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. No. 5,728,388, and International Publication No. WO 2008/081035.

The T cells of the immunotherapy can come from any source known in the art. For example, T cells can be differentiated in vitro from a hematopoietic stem cell population; induced pluripotent stem cells (iPS), embryonic stem cells (ES), or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

The term “engineered Autologous Cell Therapy,” which can be abbreviated as “eACT™,” also known as adoptive cell transfer, is a process by which a patient's own T cells are collected and subsequently genetically altered to recognize and target one or more antigens expressed on the cell surface of one or more specific tumor cells or malignancies. T cells can be engineered to express, for example, a T cell receptor (TCR).

A “patient” as used herein includes any human who is afflicted with a cancer (e.g., a lymphoma or leukemia). The terms “subject” and “patient” are used interchangeably herein.

As used herein, the term “in vitro cell” refers to any cell, which is cultured ex vivo. In particular, an in vitro cell can include a T cell.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that can comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex that specifically binds with a cognate stimulatory ligand present on an antigen present cell. A “stimulatory ligand” is a ligand that when present on an antigen presenting cell (e.g., an APC, a dendritic cell, a B-cell, and the like) can specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody, an MHC Class I molecule loaded with a peptide, a superagonist anti-CD2 antibody, and a superagonist anti-CD28 antibody.

A “costimulatory signal,” as used herein, refers to a signal, which in combination with a primary signal, such as TCR/CD3 ligation, leads to a T cell response, such as, but not limited to, proliferation and/or upregulation or down regulation of key molecules.

A “costimulatory ligand” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin β receptor, MHC class I chain-related protein A (MICA), MHC class I chain-related protein B (MICB), OX40 ligand, PD-L2, or programmed death (PD) L1. A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (α; β; delta; epsilon; γ; zeta), CD30, CD37, CD4, CD4, CD40, CD49a, CD49D, CD49f, CD5, CD64, CD69, CD7, CD80, CD83 ligand, CD84, CD86, CD8a, CD80, CD9, CD96 (Tactile), CDl-la, CDl-lb, CDl-lc, CDl-ld, CDS, CEACAMI, CRT AM, DAP-10, DNAM1 (CD226), Fc γ receptor, GADS, GITR, HVEM (LIGHTR), IA4, ICAM-1, ICAM-1, ICOS, Ig α (CD79a), IL2R β, IL2R γ, IL7R α, integrin, ITGA4, ITGA4, ITGA6, ITGAD, ITGAE, ITGAL, ITGAM, ITGAX, ITGB2, ITGB7, ITGBI, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CDl la/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Lyl08), SLAMF7, SLP-76, TNF, TNFr, TNFR2, Toll ligand receptor, TRANCE/RANKL, VLA1, or VLA-6, or fragments, truncations, or combinations thereof.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity, or recurrence of a symptom, complication or condition, or biochemical indicia associated with a disease. In one embodiment, “treatment” or “treating” includes a partial remission. In another embodiment, “treatment” or “treating” includes a complete remission.

As used herein, a “TCR proxy” is a molecule (e.g., a peptide, a protein, a synthetic molecule, etc.) that initiates downstream signaling elements that allow or facilitate the development of a T cell from a stem cell in the absence of an endogenous TCR and/or pre-TCR. In some embodiments, the TCR proxy is a defined TCR, a preTCR, a pTa monomer, a pTa/TCR β heterodimer, a TCR α molecule, a TCR β molecule, a TCR γ molecule, a TCR delta molecule, a TCR α/β heterodimer, a TCR γ/delta heterodimer, any homodimer of the previous molecules, a TCR like molecule, or other molecule that initiates a TCR signal to allow T cell development. In some embodiments, a TCR proxy comprises one or more molecules (e.g., one, two, three, four, five, six or more molecules). In some embodiments, the one or more molecules are proteins. In some embodiments, the TCR proxy is a protein complex.

As used herein, the term “selectable” means a molecule capable of being targeted by an antibody. In some embodiments, a selectable surface marker is molecule expressed on the surface that is capable of being targeted by an antigen binding molecule (e.g., an antibody).

To calculate percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span,” as determined by the algorithm.) In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Various aspects of the invention are described in further detail in the following subsections.

### DETAILED DESCRIPTION

TCR gene therapy is a promising modality to treat cancer. The present disclosure provides methods to engineer tumor-reactive T cells by the introduction of an exogenous T cell receptor gene with specificity for a tumor-antigen. Thereby, tumor-specific T cell populations are generated which are either absent or dysfunctional in cancer patients. There are two essential requirements for therapeutic TCR genes: (1) they need to mediate tumor recognition and elucidation of effector functions by TCR td T cells and (2) their specificity needs to be sufficiently restricted to avoid detrimental T cell reactivity against either allo-HLA alleles or pMHC-complexes which are not exclusively expressed at the tumor cell surface.

In the region of 5^{%} of all human cancers are HPV induced, with HPV16 being by far the most abundant cancer-associated high risk HPV genotype. Approximately, 50% of oropharyngeal cancers and cervical cancers are HPV16-associated, with many rarer urogenital cancers also being HPV16 associated. By inhibiting the function of the Rb tumor suppressor protein, the E7 protein from HPV16 promotes cellular transformation and contributes towards the maintenance of the malignant phenotype in HPV16-associated cancers. These factors together with its tumor-restricted expression make HPV16 E7 a very promising target for TCR gene therapy of HPV16-associated cancers.

However, in addition to the intrinsic properties of the therapeutic TCR genes, successful TCR gene therapy is also dependent upon high level cell surface expression of the therapeutic TCR heterodimer on TCR td T cells. For a TCRαβ heterodimer to be expressed at the surface of a T cell it has to be associated with a CD3 complex and the components of the CD3 complex are rate limiting. Therefore, in TCR td T cells the exogenous TCR chains have to compete with endogenous TCR chains for assembly with the CD3 complex and cell surface expression. Furthermore, pairing of exogenous TCR chains with endogenous TCR chains, also reduces the cell surface expression of exogenous TCRαβ heterodimers. This process, called TCR mispairing, can also lead to the formation of TCRs with deleterious self-reactive specificities. Therefore, the present disclosure provides TCR vectors designed to ensure that the TCR chains they encode can successfully compete with endogenous TCR chains for assembly with the CD3 complex and cell surface expression in TCR td T cells.

### T Cell Receptors (TCRs)

T cell receptors (TCRs) can be introduced into the vector. These engineered receptors can be readily inserted into and expressed by T cells in accordance with techniques known in the art. A TCR may be introduced to convey antigen reactivity. In some embodiments, the antigen reactivity is restricted by MHC presentation of a peptide. The TCR may be an α/β TCR, γ/delta TCR, or other. In some embodiments, the TCR is an HPV-16 E7 TCR with human constant chains (2A linked). In some embodiments, the TCR is an HPV-16 E7 TCR with murine constant chains (2A linked). In some embodiments, the chains may be linked by an IRES or any 2A family members' sequence (e.g., P2A, T2A, E2A, F2A, etc). In some embodiments, the TCR is an HPV recognizing TCR, or other viral reactive TCR (e.g., EBV, influenza, etc.). In some embodiments, a cancer or cancer associated antigen reactive TCR may be used (e.g., NYESO, MART1, gp100, etc.)

In some embodiments, the TCR is a TCR of normal/healthy peptide reactivity or other antigen reactivity/restriction. In some embodiments, the TCR is reactive against murine or other non-human MHC. In some embodiments, the TCR is a class I or class II restricted TCR.

In some embodiments, the TCR vector comprises a pMP71 retroviral vector containing a human TCR specific to a HLA-A*02:01/YMLDLQPET peptide-MHC (pMHC) complex in the following configuration: TCRβ chain-Furin cleavage site-Linker-P2A-TCRα chain. In some embodiments, the Furin cleavage site sequence is RAKR (SEQ ID NO: 20). The vector encoded TCR α and β chains are codon optimized for expression in humans. The TCR vector also contains a multiplexed miRNA cassette that targets the endogenous TCR chains in TCR td T cells for knockdown (miRαβ cassette). The miRαβ cassette contains TCR α and β specific miRNA (miR155_TRAC and AmiR_TRBC, respectively) that target the invariant constant domain of the TCR α and β chains respectively. The human TCR α and β chains encoded with the TCR vector are not targeted by these miRNA, as the DNA sequences encoding them are codon-optimized. The miRαβ cassette is incorporated into the pMP71 retroviral vector between splice donor and splice acceptor sites downstream of the 5′LTR. Expression of both TCR genes and the miRαβ is driven by the same retroviral promoter. This TCR vector is here after referred to as the pMP71-miRαβ-A*02:01/HPV16 E7₁₁₋₁₉ fHC TCR vector.

In some embodiments, the TCR chains may be linked by an IRES or any 2A family members' sequence (e.g., P2A, T2A, E2A, F2A, etc). Optionally, short linkers can form linkages between any or some of the domains of the TCR construct. In some embodiments, the short linker comprises the amino acid sequence SGSG (SEQ ID NO: 18). In some embodiments, the short linker is encoded by the polynucleotide sequence tctggaagcggc (SEQ ID NO: 19).

In some embodiments, the exogenous TCR is the TCR candidate with specificity for a HLA-A*02:01/HPV16-E71119 pMHC-complex (TCR1). The TCR was isolated from cervical infiltrating T cell material obtained from an HLA-A*02:01-positive individual with grade I/III cervical intraepithelial neoplasia who had previously received HPV16 E7 vaccination.

In some embodiments, the vector comprises a polynucleotide encoding a TCR comprising SEQ ID NO: 2-5. In some embodiments, the TCR comprises an amino acid sequence that is at least 70%, 75%, 80%, 85%, 90%, 91%, 92%, 93%, 94%, 95%, 96%, 97%, 98%, 99%, or 100% identical to any one of SEQ ID Nos: 2-5.

Amino Acid sequence of the TCR variable domains:
TCRA
(SEQ ID NO: 2)
GQNIDQPTEMTATEGAIVQINCTYQTSGFNGLFWYQQHAGEAPTFLSYNV
LDGLEEKGRFSSFLSRSKGYSYLLLKELQMKDSASYLCASVDGNNRLAFG
KGNQVVVIP
TCRB
(SEQ ID NO: 3)
DAGVTQSPTHLIKTRGQQVTLRCSPKSGHDTVSWYQQALGQGPQFIFQYY
EEEERQRGNFPDRFSGHQFPNYSSELNVNALLLGDSALYLCASSLGWRGG
RYNEQFFGPGTRLTVL
Amino Acid sequence of TCR α chain:
(SEQ ID NO: 4)
MKSLRVLLVILWLQLSWVWSQGQNIDQPTEMTATEGAIVQINCTYQTSGF
NGLFWYQQHAGEAPTFLSYNVLDGLEEKGRFSSFLSRSKGYSYLLLKELQ
MKDSASYLCASVDGNNRLAFGKGNQVVVIPDIQNPDPAVYQLRDSKSSDK
SVCLFTDFDSQTNVSQSKDSDVYITDKTVLDMRSMDFKSNSAVAWSNKSD
FACANAFNNSIIPEDTFFPSPESSCDVKLVEKSFETDTNLNFQNLSVIGF
RILLLKVAGFNLLMTLRLWSS
Amino Acid sequence of TCR β chain
(SEQ ID NO: 5)
MGPGLLCWALLCLLGAGLVDAGVTQSPTHLIKTRGQQVTLRCSPKSGHDT
VSWYQQALGQGPQFIFQYYEEEERQRGNFPDRFSGHQFPNYSSELNVNAL
LLGDSALYLCASSLGWRGGRYNEQFFGPGTRLTVLEDLKNVFPPEVAVFE
PSEAEISHTQKATLVCLATGFYPDHVELSWWVNGKEVHSGVSTDPQPLKE
QPALNDSRYCLSSRLRVSATFWQNPRNHFRCQVQFYGLSENDEWTQDRAK
PVTQIVSAEAWGRADCGFTSESYQQGVLSATILYEILLGKATLYAVLVSA
LVLMAMVKRKDSRG

### Target Antigens

MHC molecules are highly polymorphic proteins that regulate T cell responses (see, e.g., Owen et al., Kuby Immunology 7th ed. W. H. Freeman, 2012). The MHC molecules that display peptide antigens in humans are known as human leukocyte antigen (“HLA”). HLA class I molecules can be divided into several families or “supertypes” based upon their ability to bind similar repertoires of peptides. HLA supertypes include A2, A3, and B7. For a peptide to be recognized by a T cell receptor (TCR) and thus activate cytotoxic T lymphocytes (CTLs) and induce effector functions such as lysis of a target cell, e.g., a tumor cell, it must be associated with, or “presented by,” a major histocompatibility complex (MHC). Class I MHCs comprise a polymorphic α chain (also referred to as a heavy chain) and a non-polymorphic β microglobulin chain (also referred to as a light chain). The two chains are non-covalently associated with one another. Class II MHCs comprise an α and β chain, which associate with one another. Both classes of MHCs present peptide to TCRs. In humans, MHC class I comprises HLA-A, HLA-B, and HLA-C molecules and MHC class II comprises HLA-D molecules. Thus, target antigens recognized by a given TCR are typically peptides associated with a MHC molecule. Such peptides can be derived from proteins expressed on infected (e.g., HPV) or cancerous cells.

In some embodiments, the TCR targets a specific antigen or peptide fragment thereof. In some embodiments, the target antigen is a tumor antigen. In some embodiments, the TCR targets an antigenic peptide loaded on a MHC. In some embodiments, the antigen or antigenic peptide is selected from a tumor-associated antigen, such as 5T4, αfetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen (e.g., Human Papilloma virus serotype 16 (HPV16) E7 protein), human telomerase reverse transcriptase, IGFI receptor, IGF-II, IL-11Rα, IL-13R-a2, Influenza Virus-specific antigen; CD38, insulin growth factor (IGFI)-1, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp70-2, mutated p53, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin and the A1 domain of tenascin-C (TnC Al), thyroglobulin, tumor stromal antigens, vascular endothelial growth factor receptor-2 (VEGFR2), virus-specific antigen such as an HIV-specific antigen (such as HIV gpl20), as well as any derivate or variant of these markers.

### Vectors, Cells, and Pharmaceutical Compositions

Vectors

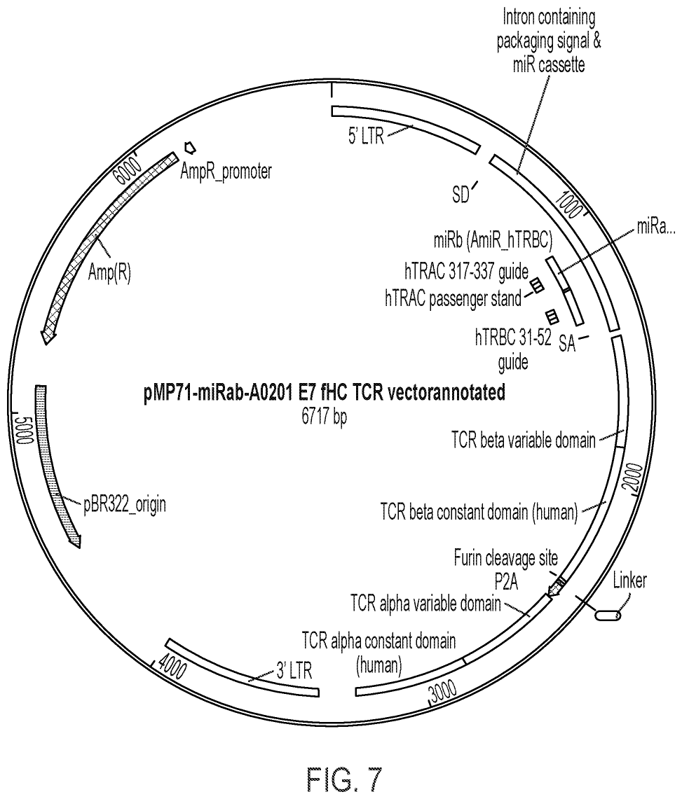

Vectors of the present disclosure introduce a therapeutic TCR into the host cell and promote a high level of surface expression. As described herein, to ensure the high-level cell surface expression of the human TCR encoded within the TCR vector on TCR td T cells, the vector comprises a multiplexed miRNA cassette that targets the endogenous TCR chains in TCR td T cells for knockdown (miRαβ cassette). According to the present disclosure, the endogenous TCR is not knocked down by siRNA. The miRαβ cassette contains TCR α and β specific miRNA (miR155_TRAC (SEQ ID NO: 7) and AmiR_TRBC (SEQ ID NO: 11), respectively) that target the invariant constant domain of the TCR α and β chains respectively. The human TCR α and β chains encoded with the TCR vector are not targeted by these miRNA, as the DNA sequences encoding them are codon-optimized. FIG. 7 depicts a vector map of the pMP71-miRαβ-A*02:01/HPV16 E7₁₁₋₁₉ fHC TCR vector.

The miRαβ cassette comprises SEQ ID NOs: 6-14.

DNA sequence of the miRαβ cassette
(SEQ ID NO: 6)
cttatcctctggctgctggaggcttgctgaaggctgtatgctgtgaaagt
ttaggttcgtatctgttttggcctctgactgacagatacgaataaacttt
cacaggacacaaggcctgttactagcactcacatggaacaaatggccaca
cgcgccaagagaacaaagtggagtattgttgcccacacccagcttccctg
gctctctgatggctcaaacacagcgagtacatgagacacgctgtgtttca
gccatcggtgagcttgggaagcatctgcagcagagcctgcctggtggccc
ctgagagattt
DNA sequence of the miR155_TRAC miRNA
(SEQ ID NO: 7)
cttatcctctggctgctggaggcttgctgaaggctgtatgctgtgaaagt
ttaggttcgtatctgttttggcctctgactgacagatacgaataaacttt
cacaggacacaaggcctgttactagcactcacatggaacaaatggccac
DNA sequence of the hTRAC 317-337 guide-strand
encoded within the miR155 TRAC miRNA
(SEQ ID NO: 8)
tgaaagtttaggttcgtatct
DNA sequence of the loop encoded within the
miR155_TRAC miRNA
(SEQ ID NO: 9)
gttttggcctctgactgac
DNA sequence of the hTRAC passenger-strand encoded
within the miR155_TRAC miRNA
(SEQ ID NO: 10)
agatacgaataaactttca
DNA sequence of the AmiR_TRBC miRNA
(SEQ ID NO: 11)
caagagaacaaagtggagtctttgttgcccacacccagcttccctggctc
tctgatggctcaaacacagcgagtacatgagacacgctgtgtttcagcca
tcggtgagcttgggaagcatctgcagcagagcctgcctggtggcccctga
gagattt
DNA sequence of the hTRBC 31-52 guide-strand
encoded within the AmiR_TRBC miRNA
(SEQ ID NO: 12)
tctgatggctcaaacacagcga
DNA sequence of the loop encoded within the AmiR_
TRBC miRNA
(SEQ ID NO: 13)
gtacatgagac
DNA sequence of the hTRBC passenger-strand encoded
within the AmiR_TRBC miRNA
(SEQ ID NO: 14)
acgctgtgtttcagccatcggt
DNA sequence of the pMP71-miRαβ-A*02:01/HPV16
E7₁₁₋₁₉ fHC TCR vector
(SEQ ID NO: 15)
ctagcttaagtaacgccattttgcaaggcatggaaaatacataactgag
aatagagaagttcagatcaaggttaggaacagagagacagcagaatatg
ggccaaacaggatatctgtggtaagcagttcctgccccggctcagggcc
aagaacagttggaacagcagaatatgggccaaacaggatatctgtggta
agcagttcctgccccggctcagggccaagaacagatggtccccagatgc
ggtcccgccctcagcagtttctagagaaccatcagatgtttccagggtg
ccccaaggacctgaaatgaccctgtgccttatttgaactaaccaatcag
ttcgcttctcgcttctgttcgcgcgcttctgctccccgagctcaataaa
agagcccacaacccctcactcggcgcgccagtcctccgatagactgcgt
cgcccgggtacccgtattcccaataaagcctcttgctgtttgcatccga
atcgtggactcgctgatccttgggagggtctcctcagattgattgactg
cccacctcgggggtattcatttggaggttccaccgagatttggagaccc
ctgcccagggaccaccgacccccccgccgggaggtaagctggccagcgg
tcgtttcgtgtctgtctctgtctttgtgcgtgtttgtgccggcatctaa
tgtttgcgcctgcgtctgtactagttggctaactagatctgtatctggc
ggtcccgcggaagaactgacgagttcgtattcccggccgcagcccctgg
gagacgtcccagcggcctcgggggcccgttttgtggcccattctgtatc
agttaacctacccgagtcggactttttggagctccgccactgtccgagg
ggtacgtggattgttgggggacgagagacagagacacttcccgcccccg
tctgaatttttgctttcggttttacgccgaaaccgcgccgcgcgtcttg
tctgctgcagcatcgttctgtgttgtctctgtctgactgtgtttctgta
tttgtctgaaaattagcacgcgccttatcctctggctgctggaggcttg
ctgaaggctgtatgctgtgaaagtttaggttcgtatctgttttggcctc
tgactgacagatacgaataaactttcacaggacacaaggcctgttacta
gcactcacatggaacaaatggccacacgcgccaagagaacaaagtggag
tctttgttgcccacacccagcttccctggctctctgatggctcaaacac
agcgagtacatgagacacgctgtgtttcagccatcggtgagcttgggaa
gcatctgcagcagagcctgcctggtggcccctgagagatttacgcgtat
tcatgcatgacaaagttaagtaatagtccctctctccaagctcacttac
aggcggccgcgccaccatgggacctggattgctttgttgggccctgctg
tgtctgcttggagctggacttgtggatgccggcgtgacacagtctccca
cacacctgatcaagaccagaggccagcaagtgaccctgagatgtagccc
taagagcggccacgacaccgtgtcttggtatcagcaggctatggccagg
gacctcagttcatcttccagtactacgaggaagaggaacggcagcgggg
caacttccctgatagattctctggccatcagttccccaactacagcagc
gagctgaacgtgaacgctctgctgctgggcgatagcgccctgtatctgt
gtgccagttctcttggttggagaggcggcagatacaacgagcagttatt
ggccctggcaccagactgaccgtgctggaagatctgaagaacgtgttcc
cacctgaggtggccgtgtttgagccttctgaggccgagatcagccacac
acagaaagccacactcgtgtgcctggccaccggcttttatcccgatcac
gtggaactgtcttggtgggtcaacggcaaagaggtgcacagcggcgtta
gcacagaccctcagcctctgaaagagcagcccgctctgaacgacagcag
atactgtctgagcagcagactgagagtgtccgccaccttctggcagaac
cccagaaaccacttcagatgccaggtgcagttctacggcctgagcgaga
atgacgagtggacccaggatagagccaagcctgtgacacagattgtgtc
tgccgaagcctggggcagagccgattgtggctttacaagcgagtcttac
cagcagggcgtgctgagcgccacaatcctgtatgagatcctgctgggca
aagccactctgtacgctgtgctggtttctgccctggtgctgatggctat
ggtcaagcggaaggatagcagaggcagagccaagagatctggaagcggc
gccacaaacttctcactgctgaaacaggccggcgacgtggaagagaacc
ctggacctatgaagtccctgcgggtgctgctggttattctgtggctgca
gctgagctgggtttggagccagggacagaacatcgaccagcctaccgag
atgacagccaccgaaggcgccatcgtgcagatcaattgcacctaccaga
ccagcggcttcaacggcctgttctggtatcaacagcatgccggcgaggc
ccctaccttcctgagctataatgtgctggacggcctggaagaaaagggc
agattcagcagcttcctgtccagaagcaagggctacagctacctgctgc
tgaaagaactccagatgaaggacagcgcctcctacctgtgtgcctccgt
ggatggaaacaacagactggccttcggcaagggcaaccaggtggtggtc
atccccgacattcagaaccctgatcctgccgtgtaccagctgagagaca
gcaagagcagcgacaagagcgtgtgtctgttcaccgacttcgacagcca
gaccaacgtgtcccagagcaaggactccgacgtgtacatcaccgacaag
acagtgctggacatgcggagcatggacttcaagagcaatagcgccgtgg
cctggtccaacaagagcgattttgcctgcgccaacgccttcaacaacag
catcatccctgaggacacattcttcccaagtcctgagagcagctgcgac
gtgaagctggtggaaaagagcttcgagacagacaccaacctgaacttcc
agaacctgagcgtgatcggcttccggatcctgcttctgaaggtggccgg
cttcaatctgctgatgacactgagactgtggtccagctgaattcggatc
caagcttaggcctgctcgctttcttgctgtcccatttctattaaaggtt
cctttgttccctaagtccaactactaaactgggggatattatgaagggc
cttgagcatctggattctgcctagcgctaagcttaacacgagccataga
tagaataaaagattttatttagtctccagaaaaaggggggaatgaaaga
ccccacctgtaggtttggcaagctagcttaagtaacgccattttgcaag
gcatggaaaatacataactgagaatagagaagttcagatcaaggttagg
aacagagagacagcagaatatgggccaaacaggatatctgtggtaagca
gttcctgccccggctcagggccaagaacagttggaacagcagaatatgg
gccaaacaggatatctgtggtaagcagttcctgccccggctcagggcca
agaacagatggtccccagatgcggtcccgccctcagcagtttctagaga
accatcagatgtttccagggtgccccaaggacctgaaatgaccctgtgc
cttatttgaactaaccaatcagttcgcttctcgcttctgttcgcgcgct
tctgctccccgagctcaataaaagagcccacaacccctcactcggcgcg
ccagtcctccgatagactgcgtcgcccgggtacccgtgttctcaataaa
ccctcttgcagttgcatccgactcgtggtctcgctgttccttgggaggg
tctcctctgagtgattgactgcccacctcgggggtctttcattctcgag
cagcttggcgtaatcatggtcatagctgtttcctgtgtgaaattgttat
ccgctcacaattccacacaacatacgagccggaagcataaagtgtaaag
cctggggtgcctaatgagtgagctaactcacattaattgcgttgcgctc
actgcccgctttccagtcgggaaacctgtcgtgccagctgcattaatga
atcggccaacgcgcggggagaggcggtttgcgtattgggcgctcttccg
cttcctcgctcactgactcgctgcgctcggtcgttcggctgcggcgagc
ggtatcagctcactcaaaggcggtaatacggttatccacagaatcaggg
gataacgcaggaaagaacatgtgagcaaaaggccagcaaaaggccagga
accgtaaaaaggccgcgttgctggcgtttttccataggctccgcccccc
tgacgagcatcacaaaaatcgacgctcaagtcagaggtggcgaaacccg
acaggactataaagataccaggcgtttccccctggaagctccctcgtgc
gctctcctgttccgaccctgccgcttaccggatacctgtccgcctttct
cccttcgggaagcgtggcgctttctcatagctcacgctgtaggtatctc
agttcggtgtaggtcgttcgctccaagctgggctgtgtgcacgaacccc
ccgttcagcccgaccgctgcgccttatccggtaactatcgtcttgagtc
caacccggtaagacacgacttatcgccactggcagcagccactggtaac
aggattagcagagcgaggtatgtaggcggtgctacagagttcttgaagt
ggtggcctaactacggctacactagaagaacagtatttggtatctgcgc
tctgctgaagccagttaccttcggaaaaagagttggtagctcttgatcc
ggcaaacaaaccaccgctggtagcggtggtttttttgtttgcaagcagc
agattacgcgcagaaaaaaaggatctcaagaagatcctttgatcttttc
tacggggtctgacgctcagtggaacgaaaactcacgttaagggattttg
gtcatgagattatcaaaaaggatcttcacctagatccttttaaattaaa
aatgaagttttaaatcaatctaaagtatatatgagtaaacttggtctga
cagttaccaatgcttaatcagtgaggcacctatctcagcgatctgtcta
tttcgttcatccatagttgcctgactccccgtcgtgtagataactacga
tacgggagggcttaccatctggccccagtgctgcaatgataccgcgaga
cccacgctcaccggctccagatttatcagcaataaaccagccagccgga
agggccgagcgcagaagtggtcctgcaactttatccgcctccatccagt
ctattaattgttgccgggaagctagagtaagtagttcgccagttaatag
tttgcgcaacgttgttgccattgctacaggcatcgtggtgtcacgctcg
tcgtttggtatggcttcattcagctccggttcccaacgatcaaggcgag
ttacatgatcccccatgttgtgcaaaaaagcggttagctccttcggtcc
tccgatcgttgtcagaagtaagttggccgcagtgttatcactcatggtt
atggcagcactgcataattctcttactgtcatgccatccgtaagatgct
tttctgtgactggtgagtactcaaccaagtcattctgagaatagtgtat
gcggcgaccgagttgctcttgcccggcgtcaatacgggataataccgcg
ccacatagcagaactttaaaagtgctcatcattggaaaacgttcttcgg
ggcgaaaactctcaaggatcttaccgctgttgagatccagttcgatgta
acccactcgtgcacccaactgatcttcagcatcttttactttcaccagc
gtttctgggtgagcaaaaacaggaaggcaaaatgccgcaaaaaagggaa
taagggcgacacggaaatgttgaatactcatactcttcctttttcaata
ttattgaagcatttatcagggttattgtctcatgagcggatacatattt
gaatgtatttagaaaaataaacaaataggggttccgcgcacatttcccc
gaaaagtgccacctgacgtctaagaaaccattattatcatgacattaac
ctataaaaataggcgtatcacgaggccctttcgtctcgcgcgtttcggt
gatgacggtgaaaacctctgacacatgcagctcccggagacggtcacag
cttgtctgtaagcggatgccgggagcagacaagcccgtcagggcgcgtc
agcgggtgttggcgggtgtcggggctggcttaactatgcggcatcagag
cagattgtactgagagtgcaccatatgcggtgtgaaataccgcacagat
gcgtaaggagaaaataccgcatcaggcgccattcgccattcaggctgcg
caactgttgggaagggcgatcggtgcgggcctcttcgctattacgccag
ctggcgaaagggggatgtgctgcaaggcgattaagttgggtaacgccag
ggttttcccagtcacgacgttgtaaaacgacggccagtgaattagtact

In some embodiments, the vector comprises SEQ ID NO: 15. In some embodiments, the vector comprises a nucleotide sequence that is at least 70%, 75%, 80%, 85%, 90%, 91%, 92%, 93%, 94%, 95%, 96%, 97%, 98%, 99%, or 100% identical to any one of SEQ ID No: 15.

In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof.

Exogenous promoters may be the human, murine, or any other species sequence of Ubiquitin C, EF1a, PGK, β-actin, etc. Promoters may use genomic in-frame versions of these sequences, fractions such as spliced out introns, introns intact, or any fractional junction of these sequences. Promoters may also be derived from viral elements, such as LTRs. Viruses of origin for promoters may be MPSV, MSGV, HTLV, HIV, etc. Spacer domains may include a throttle/chemically induced dimerizer to control expression upon addition of a small molecule in a titratable fashion.

Cells

The present disclosure provides engineered T cells and methods of generating engineered T cells comprising introducing the vector described herein into a host cell. The cell of the present invention may be obtained through any source known in the art. For example, T cells can be differentiated in vitro from a hematopoietic stem cell population, or T cells can be obtained from a subject.

Various pluripotent stem cells may be used to practice the present invention. For example, hematopoietic stem cells (HSC) in the bone marrow (also cord blood or peripheral blood) give rise, in addition to all other mature blood cells, to committed thymic progenitors. These thymic progenitors traffic to the thymus where they begin their development to mature T cells.

In some embodiments, embryonic stem (ES) or induced pluripotent stem (iPS) cells may be used. Suitable HSCs, ES cells, iPS cells and other stems cells may be cultivated immortal cell lines or isolated directly from a patient. Various methods for isolating, developing, and/or cultivating stem cells are known in the art and can be used to practice the present invention.

In some embodiments, the stem cell is an induced pluripotent stem cell (iPSC) generated from a reprogrammed T-cell. As described herein, the stem cell derived T cell can be used in an autologous or allogeneic setting for engineered immunotherapy.

In some embodiments, the cell can be an induced pluripotent stem cell (iPSC) derived from a T cell or non-T cell. The cell can be an embryonic stem cell. The cell can be a B cell, or any other cell from peripheral blood mononuclear cell isolates, hematopoietic progenitor, hematopoietic stem cell, mesenchymal stem cell, adipose stem cell, or any other somatic cell type.

T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. In certain embodiments, the cells collected by apheresis are washed to remove the plasma fraction, and placed in an appropriate buffer or media for subsequent processing. In some embodiments, the cells are washed with PBS. As will be appreciated, a washing step can be used, such as by using a semiautomated flow-through centrifuge, e.g., the Cobe™ 2991 cell processor, the Baxter CytoMate™, or the like. In some embodiments, the washed cells are resuspended in one or more biocompatible buffers, or other saline solution with or without buffer. In certain embodiments, the undesired components of the apheresis sample are removed. Additional methods of isolating T cells for a T cell therapy are disclosed in U.S. Patent Publication No. 2013/0287748, which is herein incorporated by reference in its entirety.

In certain embodiments, stem cells are isolated from PBMCs by lysing the red blood cells and depleting the monocytes, e.g., by using centrifugation through a PERCOLL™ gradient. In some embodiments, a specific subpopulation of T cells, such as CD4+, CD8+, CD28+, CD45RA⁺, and CD45RO⁺ T cells is further isolated by positive or negative selection techniques known in the art. For example, enrichment of a T cell population by negative selection can be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected can be used. For example, to enrich for CD4⁺ cells by negative selection, a monoclonal antibody cocktail typically includes antibodies to CD8, CD11b, CD14, CD16, CD20, and HLA-DR. In certain embodiments, flow cytometry and cell sorting are used to isolate cell populations of interest for use in the present invention.

In some embodiments, PBMCs are used directly for genetic modification with the immune cells (such as TCRs) using methods as described herein. In certain embodiments, after isolating the PBMCs, T lymphocytes are further isolated, and both cytotoxic and helper T lymphocytes are sorted into naive, stem cell memory, central memory, effector memory, and effector T cell subpopulations either before or after genetic modification and/or expansion.

In some embodiments, CD8⁺ cells are further sorted into naive, stem cell memory, central memory, effector memory, and effector cells by identifying cell surface antigens that are associated with each of these types of CD8⁺ cells. In some embodiments, phenotypic markers of central memory T cells include CCR7, CD3, CD28, CD45RO, CD62L, and CD127 and are negative for granzyme B. In some embodiments, central memory T cells are CD8⁺, CD45RO⁺, and CD62L⁺ T cells. In some embodiments, effector T cells are negative for CCR7, CD28, CD62L, and CD127 and positive for granzyme B and perforin. In certain embodiments, CD4⁺ T cells are further sorted into subpopulations. For example, CD4+T helper cells can be sorted into naive, central memory and effector cells by identifying cell populations that have cell surface antigens.

In some embodiments, the immune cells, e.g., T cells, are genetically modified following isolation using known methods, or the immune cells are activated and expanded (or differentiated in the case of progenitors) in vitro prior to being genetically modified. In another embodiment, the immune cells, e.g., T cells, are genetically modified with the antigen receptors described herein (e.g., transduced with a viral vector comprising one or more nucleotide sequences encoding a TCR) and then are activated and/or expanded in vitro. Methods for activating and expanding T cells are known in the art and are described, e.g., in U.S. Pat. Nos. 6,905,874; 6,867,041; and 6,797,514, and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642, and PCT Publication No. WO 2012/129514, the contents of which are hereby incorporated by reference in their entirety.

In certain embodiments, the T cells are obtained from a donor subject. In some embodiments, the donor subject is human patient afflicted with a cancer or a tumor. In other embodiments, the donor subject is a human patient not afflicted with a cancer or a tumor.

Compositions

Other aspects of the present invention are directed to compositions comprising a polynucleotide described herein, a vector described herein, a polypeptide described herein, or an in vitro cell described herein. In some embodiments, the composition comprises a pharmaceutically acceptable carrier, diluent, solubilizer, emulsifier, preservative, and/or adjuvant. In some embodiments, the composition comprises an excipient. In some embodiments, the composition comprises a polynucleotide encoding a TCR described herein. In another embodiment, the composition comprises a TCR encoded by a polynucleotide of the present invention. In some embodiments, the composition comprises a T cell comprising a TCR described herein.

In other embodiments, the composition is selected for parenteral delivery, for inhalation, or for delivery through the digestive tract, such as orally. The preparation of such pharmaceutically acceptable compositions is within the ability of one skilled in the art. In certain embodiments, buffers are used to maintain the composition at physiological pH or at a slightly lower pH, typically within a pH range of from about 5 to about 8. In certain embodiments, when parenteral administration is contemplated, the composition is in the form of a pyrogen-free, parenterally acceptable aqueous solution comprising a composition described herein, with or without additional therapeutic agents, in a pharmaceutically acceptable vehicle. In certain embodiments, the vehicle for parenteral injection is sterile distilled water in which composition described herein, with or without at least one additional therapeutic agent, is formulated as a sterile, isotonic solution, properly preserved. In certain embodiments, the preparation involves the formulation of the desired molecule with polymeric compounds (such as polylactic acid or polyglycolic acid), beads or liposomes, that provide for the controlled or sustained release of the product, which are then be delivered via a depot injection. In certain embodiments, implantable drug delivery devices are used to introduce the desired molecule.

### Cancer Treatment

The methods described herein can be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In certain embodiments, the methods induce a complete response. In other embodiments, the methods induce a partial response.

In some embodiments, the cell product may be used in oncology, immunosuppression, autoimmune control, vaccine or as a prophylactic measure. The cell may be used as a commercial product, a clinical trial, preclinical work, basic research. The cell may be used for human and/or veterinary medicine. In some embodiments, the cell product may be used as a detection reagent/discovery research.

Cancers that may be treated include tumors that are not vascularized, not yet substantially vascularized, or vascularized. The cancer may also include solid or non-solid tumors. In some embodiments, the cancer is a hematologic cancer. In some embodiments, the cancer is of the white blood cells. In other embodiments, the cancer is of the plasma cells. In some embodiments, the cancer is leukemia, lymphoma, or myeloma. In certain embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, diffuse large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBC), small cell- or a large cell-follicular lymphoma, splenic marginal zone lymphoma (SMZL), systemic amyloid light chain amyloidosis, T-cell acute lymphoid leukemia (“TALL”), T-cell lymphoma, transformed follicular lymphoma, Waldenstrom macroglobulinemia, or a combination thereof. In some embodiments, the cancer is a solid tumor, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, or a combination thereof.

In some embodiments, the methods further comprise administering a chemotherapeutic. In certain embodiments, the chemotherapeutic selected is a lymphodepleting (preconditioning) chemotherapeutic. Beneficial preconditioning treatment regimens, along with correlative beneficial biomarkers, are described in U.S. Pat. No. 9,855,298 and International Publication No. WO 2016/191755, which are hereby incorporated by reference, in their entirety herein. These describe, e.g., methods of conditioning a patient in need of a T cell therapy comprising administering to the patient specified beneficial doses of cyclophosphamide (between 200 mg/m²/day and 2000 mg/m²/day) and specified doses of fludarabine (between 20 mg/m²/day and 900 mg/m²/day). One such dose regimen involves treating a patient comprising administering daily to the patient about 500 mg/m²/day of cyclophosphamide and about 60 mg/m²/day of fludarabine for three days prior to administration of a therapeutically effective amount of engineered T cells to the patient.

In some embodiments, cyclophosphamide is administered to the patient at a dose between about 30 mg/m²/day and 2000 mg/m²/day, about 40 mg/m²/day and 2000 mg/m²/day, about 50 mg/m²/day and 2000 mg/m²/day, about 50 mg/m²/day and 1800 mg/m²/day, about 50 mg/m²/day and 1600 mg/m²/day, about 50 mg/m²/day and 1400 mg/m²/day, about 50 mg/m²/day and 1200 mg/m²/day, about 50 mg/m²/day and 1000 mg/m²/day, about 50 mg/m²/day and 900 mg/m²/day, about 50 mg/m²/day and 800 mg/m²/day, about 50 mg/m²/day and 700 mg/m²/day, about 50 mg/m²/day and 600 mg/m²/day, about 50 mg/m²/day and 500 mg/m²/day, about 50 mg/m²/day and 400 mg/m²/day, about 50 mg/m²/day and 300 mg/m²/day, about 50 mg/m²/day and 200 mg/m²/day, about 50 mg/m²/day and 100 mg/m²/day, about 60 mg/m²/day and 2000 mg/m²/day, about 70 mg/m²/day and 2000 mg/m²/day, about 80 mg/m²/day and 2000 mg/m²/day, about 90 mg/m²/day and 2000 mg/m²/day, about 100 mg/m²/day and 2000 mg/m²/day, about 120 mg/m²/day and 2000 mg/m²/day, about 150 mg/m²/day and 2000 mg/m²/day, about 200 mg/m²/day and 2000 mg/m²/day, about 300 mg/m²/day and 2000 mg/m²/day, about 400 mg/m²/day and 2000 mg/m²/day, about 500 mg/m²/day and 2000 mg/m²/day, about 600 mg/m²/day and 2000 mg/m²/day, about 700 mg/m²/day and 2000 mg/m²/day, about 800 mg/m²/day and 2000 mg/m²/day, about 900 mg/m²/day and 2000 mg/m²/day, about 100 mg/m²/day and 2000 mg/m²/day, about 1100 mg/m²/day and 2000 mg/m²/day, about 1200 mg/m²/day and 2000 mg/m²/day, about 1300 mg/m²/day and 2000 mg/m²/day, about 1400 mg/m²/day and 2000 mg/m²/day, about 1500 mg/m²/day and 2000 mg/m²/day, about 1600 mg/m²/day and 2000 mg/m²/day, about 1700 mg/m²/day and 2000 mg/m²/day, about 1800 mg/m²/day and 2000 mg/m²/day, or between about 1900 mg/m²/day and 2000 mg/m²/day.

In some embodiments, cyclophosphamide is administered to the patient at a dose of at least about 30 mg/m²/day, at least about 40 mg/m²/day, at least about 50 mg/m²/day, at least about 60 mg/m²/day, at least about 70 mg/m²/day, at least about 80 mg/m²/day, at least about 90 mg/m²/day, at least about 100 mg/m²/day, at least about 110 mg/m²/day, at least about 120 mg/m²/day, at least about 130 mg/m²/day, at least about 140 mg/m²/day, at least about 150 mg/m²/day, at least about 160 mg/m²/day, at least about 170 mg/m²/day, at least about 180 mg/m²/day, at least about 190 mg/m²/day, at least about 200 mg/m²/day, at least about 210 mg/m²/day, at least about 220 mg/m²/day, at least about 230 mg/m²/day, at least about 230 mg/m²/day, at least about 240 mg/m²/day, at least about 250 mg/m²/day, at least about 260 mg/m²/day, at least about 270 mg/m²/day, at least about 280 mg/m²/day, at least about 290 mg/m²/day, at least about 300 mg/m²/day, at least about 310 mg/m²/day, at least about 320 mg/m²/day, at least about 330 mg/m²/day, at least about 340 mg/m²/day, at least about 350 mg/m²/day, at least about 360 mg/m²/day, at least about 370 mg/m²/day, at least about 380 mg/m²/day, at least about 390 mg/m²/day, at least about 400 mg/m²/day, at least about 410 mg/m²/day, at least about 420 mg/m²/day, at least about 430 mg/m²/day, at least about 440 mg/m²/day, at least about 450 mg/m²/day, at least about 460 mg/m²/day, at least about 470 mg/m²/day, at least about 480 mg/m²/day, at least about 490 mg/m²/day, at least about 500 mg/m²/day, at least about 600 mg/m²/day, at least about 700 mg/m²/day, at least about 800 mg/m²/day, at least about 900 mg/m²/day, at least about 1000 mg/m²/day, at least about 1100 mg/m²/day, at least about 1200 mg/m²/day, at least about 1300 mg/m²/day, at least about 1400 mg/m²/day, at least about 1500 mg/m²/day, at least about 1600 mg/m²/day, at least about 1700 mg/m²/day, at least about 1800 mg/m²/day, or at least about 1900 mg/m²/day.

In some embodiments fludarabine is administered at a dose between about 10 mg/m²/day and 900 mg/m²/day, about 10 mg/m²/day and 800 mg/m²/day, about 10 mg/m²/day and 700 mg/m²/day, about 10 mg/m²/day and 600 mg/m²/day, about 10 mg/m²/day and 500 mg/m²/day, about 10 mg/m²/day and 400 mg/m²/day, about 10 mg/m²/day and 300 mg/m²/day, about 10 mg/m²/day and 200 mg/m²/day, about 10 mg/m²/day and 100 mg/m²/day, about 10 mg/m²/day and 90 mg/m²/day, about 10 mg/m²/day and 80 mg/m²/day, about 10 mg/m²/day and 70 mg/m²/day, about 10 mg/m²/day and 60 mg/m²/day, about 10 mg/m²/day and 50 mg/m²/day, about 10 mg/m²/day and 40 mg/m²/day, about 10 mg/m²/day and 30 mg/m²/day, about 10 mg/m²/day and 20 mg/m²/day, about 10 mg/m²/day and 15 mg/m²/day, about 20 mg/m²/day and 900 mg/m²/day, about 30 mg/m²/day and 900 mg/m²/day, about 40 mg/m²/day and 900 mg/m²/day, about 50 mg/m²/day and 900 mg/m²/day, about 60 mg/m²/day and 900 mg/m²/day, about 70 mg/m²/day and 900 mg/m²/day, about 80 mg/m²/day and 900 mg/m²/day, about 90 mg/m²/day and 900 mg/m²/day, about 100 mg/m²/day and 900 mg/m²/day, about 150 mg/m²/day and 900 mg/m²/day, about 200 mg/m²/day and 900 mg/m²/day, about 250 mg/m²/day and 900 mg/m²/day, about 300 mg/m²/day and 900 mg/m²/day, about 400 mg/m²/day and 900 mg/m²/day, about 500 mg/m²/day and 900 mg/m²/day, about 550 mg/m²/day and 900 mg/m²/day, about 600 mg/m²/day and 900 mg/m²/day, about 650 mg/m²/day and 900 mg/m²/day, about 700 mg/m²/day and 900 mg/m²/day, or between about 800 mg/m²/day and 900 mg/m²/day.

In some embodiments, fludarabine is administered at least about 30 mg/m²/day, at least about 40 mg/m²/day, at least about 50 mg/m²/day, at least about 60 mg/m²/day, at least about 70 mg/m²/day, at least about 80 mg/m²/day, at least about 90 mg/m²/day, at least about 100 mg/m²/day, at least about 110 mg/m²/day, at least about 120 mg/m²/day, at least about 130 mg/m²/day, at least about 140 mg/m²/day, at least about 150 mg/m²/day, at least about 160 mg/m²/day, at least about 170 mg/m²/day, at least about 180 mg/m²/day, at least about 190 mg/m²/day, at least about 200 mg/m²/day, at least about 210 mg/m²/day, at least about 220 mg/m²/day, at least about 230 mg/m²/day, at least about 230 mg/m²/day, at least about 240 mg/m²/day, at least about 250 mg/m²/day, at least about 260 mg/m²/day, at least about 270 mg/m²/day, at least about 280 mg/m²/day, at least about 290 mg/m²/day, at least about 300 mg/m²/day, at least about 310 mg/m²/day, at least about 320 mg/m²/day, at least about 330 mg/m²/day, at least about 340 mg/m²/day, at least about 350 mg/m²/day, at least about 360 mg/m²/day, at least about 370 mg/m²/day, at least about 380 mg/m²/day, at least about 390 mg/m²/day, at least about 400 mg/m²/day, at least about 410 mg/m²/day, at least about 420 mg/m²/day, at least about 430 mg/m²/day, at least about 440 mg/m²/day, at least about 450 mg/m²/day, at least about 460 mg/m²/day, at least about 470 mg/m²/day, at least about 480 mg/m²/day, at least about 490 mg/m²/day, at least about 500 mg/m²/day, at least about 600 mg/m²/day, at least about 700 mg/m²/day, at least about 800 mg/m²/day, at least about 900 mg/m²/day.

In some embodiments, cyclophosphamide and fludarabine are administered daily at specified doses. In some embodiments, cyclophosphamide is administered at a dose of about 60 mg/m²/day and fludarabine is administered at a dose of about 25 mg/m²/day. In some embodiments, cyclophosphamide is administered at a dose of about 300 mg/m²/day and fludarabine is administered at a dose of about 30 mg/m²/day. In some embodiments, cyclophosphamide is administered at a dose of about 500 mg/m²/day and fludarabine is administered at a dose of about 30 mg/m²/day.

In some embodiments, the cyclophosphamide and fludarabine regimen is administered at a specified dose for two days prior to administration of a therapeutically effective amount of engineered T cells to the patient. In some embodiments, the cyclophosphamide and fludarabine regimen is administered at a specified dose for three days prior to administration of a therapeutically effective amount of engineered T cells to the patient. In some embodiments, the cyclophosphamide and fludarabine regimen is administered at a specified dose for four days prior to administration of a therapeutically effective amount of engineered T cells to the patient. In some embodiments, the cyclophosphamide and fludarabine regimen is administered at a specified dose for five days prior to administration of a therapeutically effective amount of engineered T cells to the patient. In some embodiments, the cyclophosphamide and fludarabine regimen is administered at a specified dose within one week prior to administration of a therapeutically effective amount of engineered T cells to the patient. In some embodiments, the cyclophosphamide and fludarabine regimen is administered at a specified dose within 5 days prior to administration of a therapeutically effective amount of engineered T cells to the patient. In some embodiments, the cyclophosphamide and fludarabine regimen is administered at a specified dose within 4 days prior to administration of a therapeutically effective amount of engineered T cells to the patient.

In other embodiments, the antigen binding molecule, transduced (or otherwise engineered) cells (e.g., comprising a TCR), and the chemotherapeutic agent are administered each in an amount effective to treat the disease or condition in the subject.

In certain embodiments, compositions comprising TCR-expressing immune effector cells disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb) and doxetaxel (TAXOTERE®, Rhone-Poulene Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. In some embodiments, compositions comprising CAR- and/or TCR-expressing immune effector cells disclosed herein may be administered in conjunction with an anti-hormonal agent that acts to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In some embodiments, the chemotherapeutic agent is administered at least 1 month before administering the cell or nucleic acid. In some embodiments, the methods further comprise administering two or more chemotherapeutic agents.

A variety of additional therapeutic agents may be used in conjunction with the compositions described herein. For example, potentially useful additional therapeutic agents include PD-1 inhibitors such as nivolumab (OPDIVO®), pembrolizumab (KEYTRUDA®), pembrolizumab, pidilizumab (CureTech), and atezolizumab (Roche).

Additional therapeutic agents suitable for use in combination with the invention include, but are not limited to, ibrutinib (IMBRUVICA®), ofatumumab (ARZERRA®), rituximab (RITUXAN®), bevacizumab (AVASTIN®), trastuzumab (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In additional embodiments, the composition comprising TCR-containing immune cells are administered with an anti-inflammatory agent. Anti-inflammatory agents or drugs can include, but are not limited to, steroids and glucocorticoids (including βmethasone, budesonide, dexamethasone, hydrocortisone acetate, hydrocortisone, hydrocortisone, methylprednisolone, prednisolone, prednisone, and triamcinolone), nonsteroidal anti-inflammatory drugs (NSAIDS) including aspirin, ibuprofen, naproxen, methotrexate, sulfasalazine, leflunomide, anti-TNF medications, cyclophosphamide, and mycophenolate. Exemplary NSAIDs include ibuprofen, naproxen, naproxen sodium, Cox-2 inhibitors, and sialylates. Exemplary analgesics include acetaminophen, oxycodone, and tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular), and minocycline.

In certain embodiments, the compositions described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, β, and γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; IL-15, a tumor necrosis factor such as TNF-α or TNF-β; and other polypeptide factors including LIF and kit ligand (KL). As used herein, the term cytokine includes proteins from natural sources or from recombinant cell culture, and biologically active equivalents of the native sequence cytokines.

Another aspect of the present invention is directed to a method of inducing immunity against a tumor comprising administering to a subject an effective amount of an engineered T cell disclosed herein. Another aspect of the present invention is directed to a method of inducing an immune response in a subject comprising administering an effective amount of the engineered immune cells of the present application. In some embodiments, the immune response is a T cell-mediated immune response. In some embodiments, the T cell-mediated immune response is directed against one or more target cells. In some embodiments, the engineered immune cell comprises a TCR, wherein the TCR is encoded in a miRαβ cassette containing TCR α and β specific miRNA described in the present disclosure. In some embodiments, the target cell is a tumor cell.

Another aspect of the present invention is directed to a method for treating or preventing a malignancy, said method comprising administering to a subject in need thereof an effective amount of at least one immune cell, wherein the immune cell comprises at least one TCR.

Another aspect of the present invention is directed to a method of treating a cancer in a subject in need thereof comprising administering to the subject a polynucleotide, a vector, a TCR, a cell, or a composition disclosed herein. In some embodiments, the method comprises administering a polynucleotide encoding a TCR. In some embodiments, the method comprises administering a vector comprising a polynucleotide encoding a TCR. In another embodiment, the method comprises administering a TCR encoded by a polynucleotide disclosed herein. In another embodiment, the method comprises administering a cell comprising the polynucleotide, or a vector comprising the polynucleotide, encoding a TCR.

In some embodiments, the donor T cells for use in the T cell therapy are obtained from the patient (e.g., for an autologous T cell therapy). In other embodiments, the donor stem cells to be differentiated into T cells for use in the T cell therapy are obtained from a subject that is not the patient.

The T cells can be administered at a therapeutically effective amount. For example, a therapeutically effective amount of the T cells can be at least about 10⁴ cells, at least about 10⁵ cells, at least about 10⁶ cells, at least about 10⁷ cells, at least about 10⁸ cells, at least about 109, or at least about 10¹⁰. In another embodiment, the therapeutically effective amount of the T cells is about 10⁴ cells, about 10⁵ cells, about 10⁶ cells, about 10⁷ cells, or about 10⁸ cells. In one particular embodiment, the therapeutically effective amount of the TCR T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

The present invention is further illustrated by the following examples, which should not be construed as further limiting. The contents of all references cited throughout this application are expressly incorporated herein by reference.

### EXAMPLES

### Example 1: TCR Expression in Primary Human Peripheral Mononuclear Cells (PBMC)

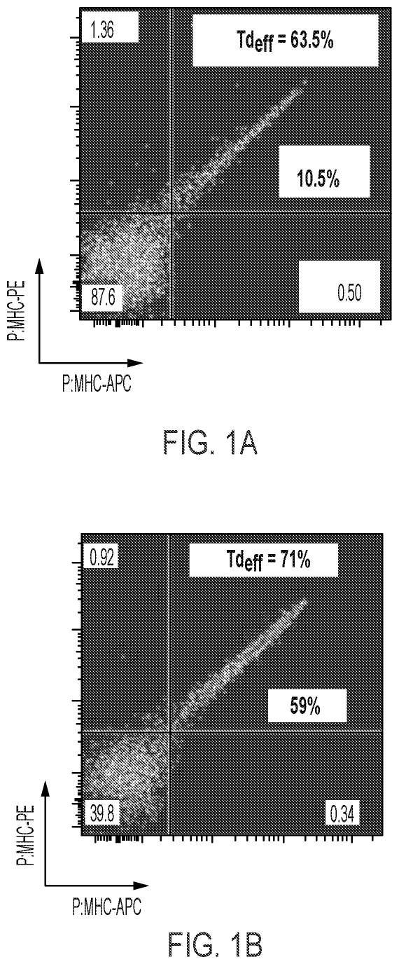

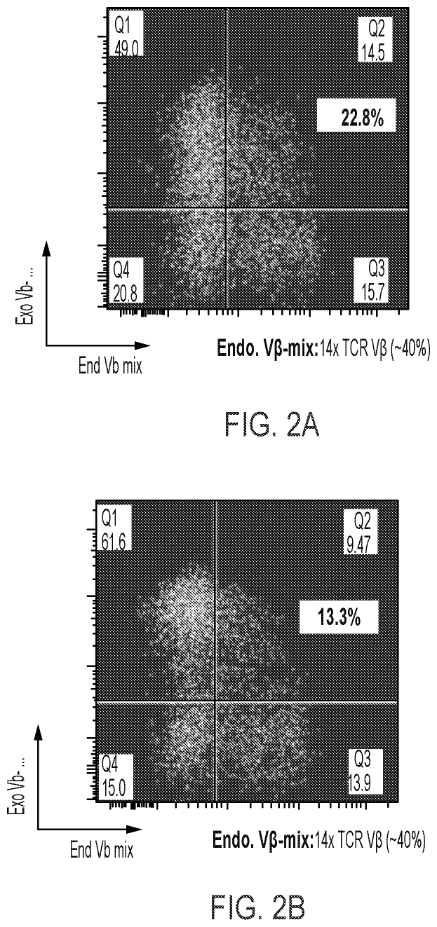

This example illustrates expression of the TCR in primary human Peripheral Mononuclear Cells (PBMC) following transduction (td) with the miRαβ TCR vector (SEQ ID NO: 15). Expression of the TCR candidate in T cells was evaluated by flow cytometry using staining with HLA-A*02:01/HPV16 E7₁₁₋₁₉ MHC-multimers (FIGS. 1A and 1B). MHC-multimer staining of CD8+ T cells was only 10.5% in T cells td with a pMP71-A*02:01/HPV16 E7₁₁₋₁₉ fHC TCR vector lacking the miRαβ cassette (FIG. 1A) compared to 59% in T cells td with the miRαβ TCR vector (SEQ ID NO: 15). (FIG. 1B). However, the markedly lower A*02:01/HPV16 E71-19-specific TCR expression on T cells td with the pMP71-A*02:01/HPV16 E71-19 fHC TCR vector could not be explained by differences in transduction efficiencies. Since staining with an antibody specific for the TCR Vβ5.6*01 gene element used by the TCR demonstrated that td efficiencies were broadly comparable in T cells td with the pMP71-A*02:01/HPV16 E71119 fHC TCR vector as compared to the miRαβ TCR vector (SEQ ID NO: 15) (63.5% of total CD8+ T cells vs 71% of total CD8+ T cells, respectively) (FIGS. 1A, 1B and FIGS. 2A, 2B ‘Exo Vb positive cells’ in Q1 and Q2). Taken together, these data demonstrate that incorporation of a miRαβ cassette in the pMP71-A*02:01/HPV16 E7₁₁₋₁₉ fHC TCR (e.g., SEQ ID NO: 15) vector leads to high level expression of the A*02:01/HPV16 E7₁₁₋₁₉ human TCR on td T cells td.

### Example 2: Reduced Endogenous TCR Expression on T Cells Transduced with the miRαβ TCR Vector

Expression of TCR α heterodimers containing endogenous TCR β chains on TCR td T cells was evaluated by flow cytometry using staining with antibodies specific for the TCR Vβ5.6*01 gene element used by the A*02:01/HPV16 E7₁₁₋₁₉ TCR and a pool of 14 different TCR Vβ specific antibodies utilized by approximately 40% of endogenous TCR β chains. This revealed that the percentage of TCR td T cells expressing some TCR α heterodimers containing an endogenous TCR β chain was markedly reduced with the use of the miRαβ TCR vector (SEQ ID NO: 15) (13.3%) compared to the pMP71-A*02:01/HPV16 E7₁₁₋₁₉ fHC TCR vector (22.8%). These data are consistent with the notion that incorporation of a miRαβ cassette in the pMP71-A*02:01/HPV16 E7₁₁₋₁₉ fHC TCR vector leads to reduced endogenous TCR expression on T cells td with this TCR.

### Example 3: Target Sensitivity of T Cells Td with the miRαβ TCR Vector

In order to determine the sensitivity of the TCR candidate, TCR td T cells were co-incubated with peptide-loaded target cells and intracellular IFN-γ levels were measured by flow cytometry. The assay was performed with 90 minutes of peptide-loading of T2 target cells at indicated concentrations and 6 h co-culture with subsequent intracellular cytokine staining (ICCS) for IFN-γ.

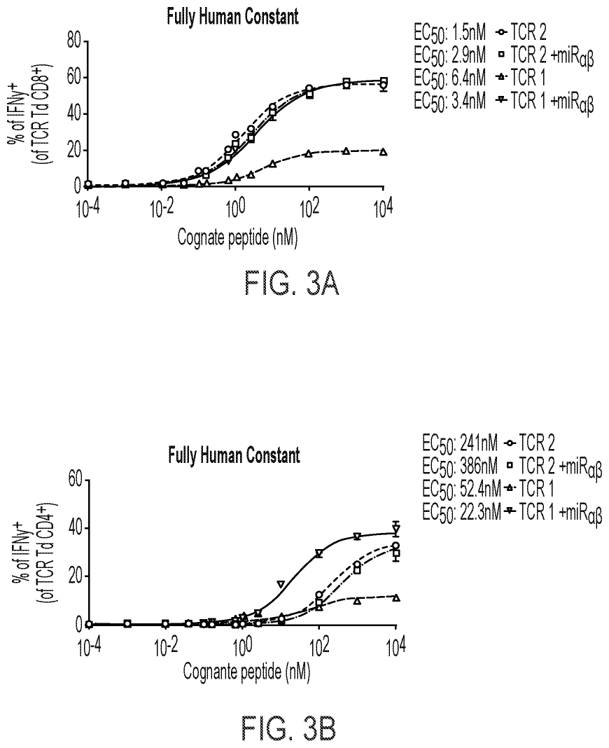

In the case of CD8+ T cells, these experiments resulted in an EC₅₀ value of approx. 3.4 nM for T cells td with the miRαβ TCR vector (SEQ ID NO: 15) vector as compared to a reduced EC₅₀ value of approx. 6.4 nM for T cells td with the TCR vector lacking the miRαβ cassette (FIG. 3A). Likewise for CD4+ T cells, a reduced EC₅₀ value of approx. 52.4 nM was observed for T cells td with the TCR vector lacking the miRαβ cassette compared to an EC₅₀ value of approx. 22.3 nM for T cells td with the pMP71-miRαβ A*02:01/IHPV16 E7₁₁₋₁₉ fHC TCR vector (FIG. 3B). Furthermore, the percentage of both CD8+ and CD4+ TCR td T cells producing IFN-γ was significantly reduced with the TCR vector lacking the miRαβ cassette.

### Example 4: Enhanced In Vitro Tumor Recognition by T Cells Td with the miRαβ TCR Vector

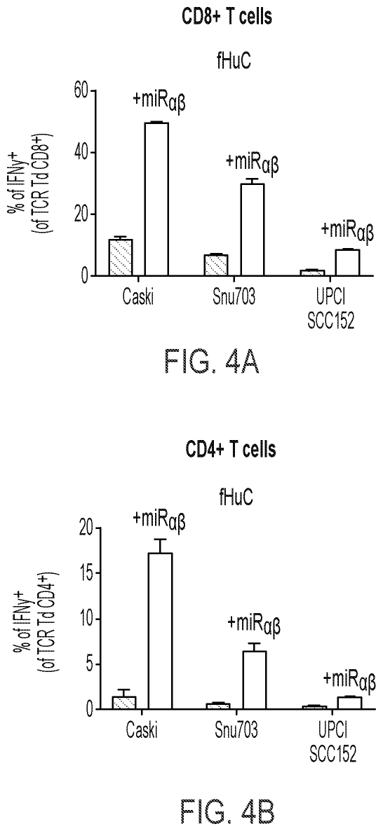

To compare in vitro tumor recognition by T cells td with the miRαβ TCR vector (SEQ ID NO: 15) versus T cells td with the TCR vector lacking the miRαβ cassette, a panel of 3 tumor cell lines was used. All 3 lines express HPV16 E7 and HLA-A*02:01. Tumor target cells were pre-seeded 24 hours prior to 6 hour co-culture with TCR td T cells. Intracellular IFN-γ levels were measured by flow cytometry. For both CD8+ and CD4+ TCR td T cells, tumor recognition by TCR Td T cells was markedly increased with miRαβ TCR vector (SEQ ID NO: 15) as compared to the TCR vector lacking the miRαβ cassette (FIGS. 4A and 4B).

### Example 5: Equivalent In Vitro and In Vivo Activity of T Cells Td with the miRαβ TCR Vector

The performance of the A*02:01/HPV16 E7₁₁₋₁₉ TCR was compared in the miRαβ TCR vector (SEQ ID NO: 15) versus a TCR vector which is being utilized in clinical studies with this TCR (clinical TCR vector ‘pMSGV fMC-Cys-LVLα TCR vector’). The pMSGV fMC-Cys-LVLα TCR vector comprises a pMSGV1 retroviral vector containing the TCR in the following configuration: TCR β chain-Furin cleavage site-Linker-P2A-TCR α chain and the TCR is codon optimized for expression in humans. This TCR vector utilizes the following modifications of the A*02:01/HPV16 E7₁₁₋₁₉-specific TCR on td T cells: (1) the human TCR constant regions (HC) are exchanged for mouse TCR constant regions (MC); (2) a cysteine is substituted in place of Thr48 of the α-chain constant domain and Ser57 of the β-chain constant domain to generate an additional interchain disulfide bond; and (3) hydrophobic substitutions to the α-chain transmembrane are introduced (sequence changed from ¹¹LSVMGLRIL¹⁹ (SEQ ID NO: 16) to ¹¹LLVIVLRIL¹⁹ (SEQ ID NO: 17))

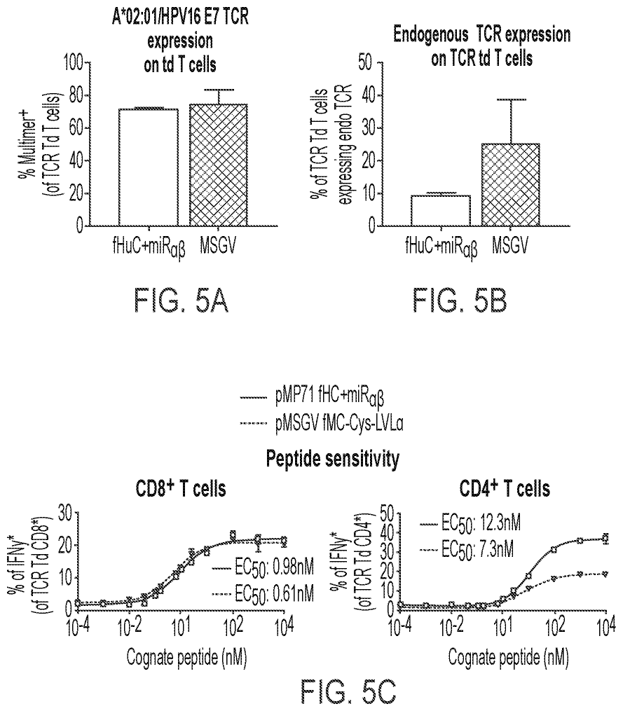

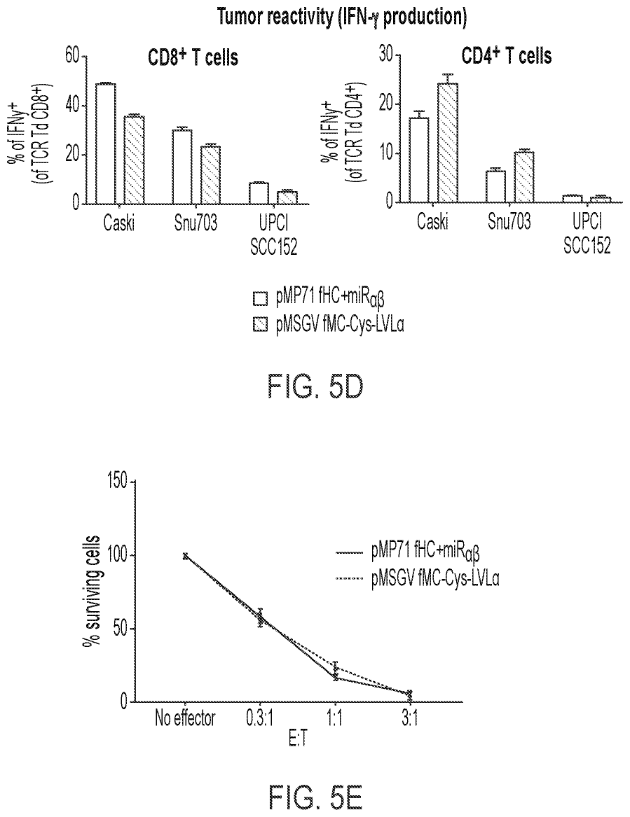

The two TCR vectors were compared in a series of in vitro studies. The miRαβ TCR vector (SEQ ID NO: 15) reduced the expression of TCR αβ heterodimers containing endogenous TCR β chains on TCR td T cells (FIG. 5B). The miRαβ TCR vector (SEQ ID NO: 15) performed at least as well as the clinical TCR vector with respect to expression of the A*02:01/HPV16 E7₁₁₋₁₉-specific TCR on td T cells (FIG. 5A), antigen sensitivity of CD8+ and CD4+ TCR td T cells (FIG. 5C); tumor-reactivity as measured by IFN-γ production in CD8+ and CD4+ TCR td T cells (FIG. 5D) and cytotoxicity of TCR td T cells (FIG. 5E).

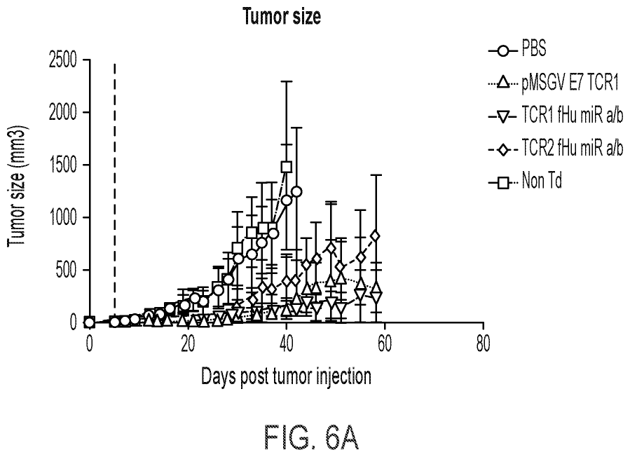

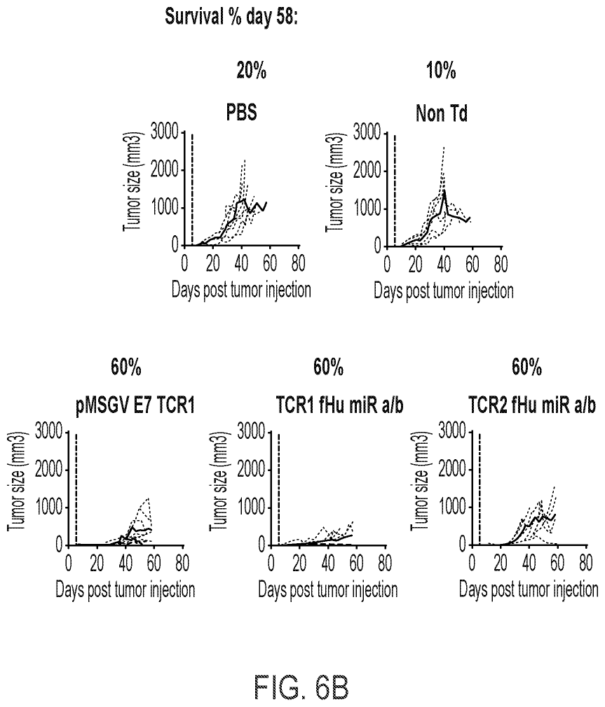

Furthermore, the ability of human T cells transduced with these two different TCR vectors to control the outgrowth of HPV16+/HLA-A*02:01 human Caski tumor cells in NSG mice was tested. Four days after tumor injection, human T cells transduced with the different TCR vector cassettes were transferred. This revealed that the T cells transduced with either TCR vector showed an equivalent ability to control tumor outgrowth (FIG. 6A and FIG. 6B).

### Sequences and SEQ ID NOs

The instant disclosure comprises a number of nucleic acid and polypeptide sequences. For convenience, Table 1 below correlates each sequence with its appropriate description and SEQ ID NO.

TABLE 1
Sequences and SEQ ID NOs.

| SEQ ID NO | Sequence | Description |
|---|---|---|
| SEQ ID NO: | YMLDLQPET | HPV16 E7 protein (AAs |
| 1 | | 11-19) expressed in |
| | | various HPV16- |
| | | asociated tumor cells |
| SEQ ID NO: | GQNIDQPTEMTATEGAIVQINCTYQTSGFNGLF | TCRα variable domain |
| 2 | WYQQHAGEAPTFLSYNVLDGLEEKGRFSSFLS | AA |
| | RSKGYSYLLLKELQMKDSASYLCASVDGNNRL | |
| | AFGKGNQVVVIP | |
| SEQ ID NO: | DAGVTQSPTHLIKTRGQQVTLRCSPKSGHDTVS | TCRβ variable domain |
| 3 | WYQQALGQGPQFIFQYYEEEERQRGNFPDRFS | AA |
| | GHQFPNYSSELNVNALLLGDSALYLCASSLGW | |
| | RGGRYNEQFFGPGTRLTVL | |
| SEQ ID NO: | MKSLRVLLVILWLQLSWVWSQGQNIDQPTEM | Amino Acid sequence of |
| 4 | TATEGAIVQINCTYQTSGFNGLFWYQQHAGEA | TCR alpha chain |
| | PTFLSYNVLDGLEEKGRFSSFLSRSKGYSYLLL | |
| | KELQMKDSASYLCASVDGNNRLAFGKGNQVV | |
| | VIPDIQNPDPAVYQLRDSKSSDKSVCLFTDFDS | |
| | QTNVSQSKDSDVYITDKTVLDMRSMDFKSNSA | |
| | VAWSNKSDFACANAFNNSIIPEDTFFPSPESSCD | |
| | VKLVEKSFETDTNLNFQNLSVIGFRILLLKVAG | |
| | FNLLMTLRLWSS | |
| SEQ ID NO: | MGPGLLCWALLCLLGAGLVDAGVTQSPTHLIK | Amino Acid sequence of |
| 5 | TRGQQVTLRCSPKSGHDTVSWYQQALGQGPQ | TCR beta chain |
| | FIFQYYEEEERQRGNFPDRFSGHQFPNYSSELN | |
| | VNALLLGDSALYLCASSLGWRGGRYNEQFFGP | |
| | GTRLTVLEDLKNVFPPEVAVFEPSEAEISHTQK | |
| | ATLVCLATGFYPDHVELSWWVNGKEVHSGVS | |
| | TDPQPLKEQPALNDSRYCLSSRLRVSATFWQNP | |
| | RNHFRCQVQFYGLSENDEWTQDRAKPVTQIVS | |
| | AEAWGRADCGFTSESYQQGVLSATILYEILLGK | |
| | ATLYAVLVSALVLMAMVKRKDSRG | |
| SEQ ID NO: | cttatcctctggctgctggaggcttgctgaaggctgtatgctgtgaaagttta | DNA sequence of the |
| 6 | ggttcgtatctgttttggcctctgactgacagatacgaataaactttcacagg | miRαβ cassette |
| | acacaaggcctgttactagcactcacatggaacaaatggccacacgcgcc | |
| | aagagaacaaagtggagtattgttgcccacacccagatccctggctctct | |
| | gatggctcaaacacagcgagtacatgagacacgctgtgtttcagccatcg | |
| | gtgagcttgggaagcatctgcagcagagcctgcctggtggcccctgaga | |
| | gattt | |
| SEQ ID NO: | cttatcctctggctgctggaggcttgctgaaggctgtatgctgtgaaagttta | DNA sequence of the |
| 7 | ggttcgtatctgttttggcctctgactgacagatacgaataaactttcacagg | miR155_TRAC miRNA |
| | acacaaggcctgttactagcactcacatggaacaaatggccac | |
| SEQ ID NO: | Tgaaagtttaggttcgtatct | DNA sequence of the |
| 8 | | hTRAC 317-337 guide- |
| | | strand encoded within |
| | | the miR155_TRAC |
| | | miRNA |
| SEQ ID NO: | gttttggcctctgactgac | DNA sequence of the |
| 9 | | loop encoded within the |
| | | miR155_TRAC miRNA |
| SEQ ID NO: | agatacgaataaactttca | DNA sequence of the |
| 10 | | hTRAC passenger- |
| | | strand encoded within |
| | | the miR155_TRAC |
| | | miRNA |
| SEQ ID NO: | caagagaacaaagtggagtctttgttgcccacacccagcttccctggctct | DNA sequence of the |
| 11 | ctgatggctcaaacacagcgagtacatgagacacgctgtgtttcagccatc | AmiR TRBC_miRNA |
| | ggtgagcttgggaagcatctgcagcagagcctgcctggtggcccctgag | |
| | agattt | |
| SEQ ID NO: | tctgatggctcaaacacagcga | DNA sequence of the |
| 12 | | hTRBC 31-52 guide- |
| | | strand encoded within |
| | | the AmiR_TRBC |
| | | miRNA |
| SEQ ID NO: | gtacatgagac | DNA sequence of the |
| 13 | | loop encoded within the |
| | | AmiR TRBC_miRNA |
| SEQ ID NO: | acgctgtgtttcagccatcggt | DNA sequence of the |
| 14 | | hTRBC passenger- |
| | | strand encoded within |
| | | the AmiR_TRBC |
| | | miRNA |
| SEQ ID NO: | ctagcttaagtaacgccattttgcaaggcatggaaaatacataactgagaat | DNA sequence of the |
| 15 | agagaagttcagatcaaggttaggaacagagagacagcagaatatgggc | pMP71-miRαβ-NCI |
| | caaacaggatatctgtggtaagcagttcctgccccggctcagggccaaga | A*02:01/HPV16 E7₁₁₋₁₉ |
| | acagttggaacagcagaatatgggccaaacaggatatctgtggtaagcag | fHC TCR vector |
| | ttcctgccccggctcagggccaagaacagatggtccccagatgcggtcc | |
| | cgccctcagcagtttctagagaaccatcagatgtttccagggtgccccaag | |
| | gacctgaaatgaccctgtgccttatttgaactaaccaatcagttcgcttctcg | |
| | cttctgttcgcgcgcttctgctccccgagctcaataaaagagcccacaacc | |
| | cctcactcggcgcgccagtcctccgatagactgcgtcgcccgggtaccc | |
| | gtattcccaataaagcctcttgctgtttgcatccgaatcgtggactcgctgat | |
| | ccttgggagggtctcctcagattgattgactgcccacctcgggggtctttca | |
| | tttggaggttccaccgagatttggagacccctgcccagggaccaccgacc | |
| | cccccgccgggaggtaagctggccagcggtcgtttcgtgtctgtctctgtc | |
| | tttgtgcgtgtttgtgccggcatctaatgtttgcgcctgcgtctgtactagttg | |
| | gctaactagatctgtatctggcggtcccgcggaagaactgacgagttcgta | |
| | ttcccggccgcagcccctgggagacgtcccagcggcctcgggggcccg | |
| | ttttgtggcccattctgtatcagttaacctacccgagtcggactttttggagct | |
| | ccgccactgtccgaggggtacgtggattgttgggggacgagagacaga | |
| | gacacttcccgcccccgtctgaatttttgctttcggttttacgccgaaaccgc | |
| | gccgcgcgtcttgtctgctgcagcatcgttctgtgttgtctctgtctgactgtg | |
| | tttctgtatttgtctgaaaattagcacgcgccttatcctctggctgctggaggc | |
| | ttgctgaaggctgtatgctgtgaaagtttaggttcgtatctgttttggcctctg | |
| | actgacagatacgaataaactttcacaggacacaaggcctgttactagcac | |
| | tcacatggaacaaatggccacacgcgccaagagaacaaagtggagtcttt | |
| | gttgcccacacccagcttccctggctctctgatggctcaaacacagcgagt | |
| | acatgagacacgctgtgtttcagccatcggtgagcttgggaagcatctgca | |
| | gcagagcctgcctggtggcccctgagagatttacgcgtattcatgcatgac | |
| | aaagttaagtaatagtccctctctccaagctcacttacaggcggccgcgcc | |
| | accatgggacctggattgctttgttgggccctgctgtgtctgcttggagctg | |
| | gacttgtggatgccggcgtgacacagtctcccacacacctgatcaagacc | |
| | agaggccagcaagtgaccctgagatgtagccctaagagcggccacgac | |
| | accgtgtcttggtatcagcaggctcttggccagggacctcagttcatcttcc | |
| | agtactacgaggaagaggaacggcagcggggcaacttccctgatagatt | |
| | ctctggccatcagttccccaactacagcagcgagctgaacgtgaacgctct | |
| | gctgctgggcgatagcgccctgtatctgtgtgccagttctcttggttggaga | |
| | ggcggcagatacaacgagcagttctttggccctggcaccagactgaccgt | |
| | gctggaagatctgaagaacgtgttcccacctgaggtggccgtgtttgagcc | |
| | ttctgaggccgagatcagccacacacagaaagccacactcgtgtgcctgg | |
| | ccaccggcttttatcccgatcacgtggaactgtcttggtgggtcaacggca | |
| | aagaggtgcacagcggcgttagcacagaccctcagcctctgaaagagca | |
| | gcccgctctgaacgacagcagatactgtctgagcagcagactgagagtgt | |
| | ccgccaccttctggcagaaccccagaaaccacttcagatgccaggtgca | |
| | gttctacggcctgagcgagaatgacgagtggacccaggatagagccaag | |
| | cctgtgacacagattgtgtctgccgaagcctggggcagagccgattgtgg | |
| | ctttacaagcgagtcttaccagcagggcgtgctgagcgccacaatcctgta | |
| | tgagatcctgctgggcaaagccactctgtacgctgtgctggtttctgccctg | |
| | gtgctgatggctatggtcaagcggaaggatagcagaggcagagccaag | |
| | agatctggaagcggcgccacaaacttctcactgctgaaacaggccggcg | |
| | acgtggaagagaaccctggacctatgaagtccctgcgggtgctgctggtt | |
| | attctgtggctgcagctgagctgggtttggagccagggacagaacatcga | |
| | ccagcctaccgagatgacagccaccgaaggcgccatcgtgcagatcaat | |
| | tgcacctaccagaccagcggcttcaacggcctgttctggtatcaacagcat | |
| | gccggcgaggcccctaccttcctgagctataatgtgctggacggcctgga | |
| | agaaaagggcagattcagcagcttcctgtccagaagcaagggctacagc | |
| | tacctgctgctgaaagaactccagatgaaggacagcgcctcctacctgtgt | |
| | gcctccgtggatggaaacaacagactggccttcggcaagggcaaccag | |
| | gtggtggtcatccccgacattcagaaccctgatcctgccgtgtaccagctg | |
| | agagacagcaagagcagcgacaagagcgtgtgtctgttcaccgacttcg | |
| | acagccagaccaacgtgtcccagagcaaggactccgacgtgtacatcac | |
| | cgacaagacagtgctggacatgcggagcatggacttcaagagcaatagc | |
| | gccgtggcctggtccaacaagagcgattttgcctgcgccaacgccttcaa | |
| | caacagcatcatccctgaggacacattcttcccaagtcctgagagcagctg | |
| | cgacgtgaagctggtggaaaagagcttcgagacagacaccaacctgaac | |
| | ttccagaacctgagcgtgatcggcttccggatcctgcttctgaaggtggcc | |
| | ggcttcaatctgctgatgacactgagactgtggtccagctgaattcggatcc | |
| | aagcttaggcctgctcgctttcttgctgtcccatttctattaaaggttcctttgtt | |
| | ccctaagtccaactactaaactgggggatattatgaagggccttgagcatct | |
| | ggattctgcctagcgctaagcttaacacgagccatagatagaataaaagat | |
| | tttatttagtctccagaaaaaggggggaatgaaagaccccacctgtaggttt | |
| | ggcaagctagcttaagtaacgccattttgcaaggcatggaaaatacataac | |
| | tgagaatagagaagttcagatcaaggttaggaacagagagacagcagaa | |
| | tatgggccaaacaggatatctgtggtaagcagttcctgccccggctcagg | |
| | gccaagaacagttggaacagcagaatatgggccaaacaggatatctgtg | |
| | gtaagcagttcctgccccggctcagggccaagaacagatggtccccaga | |
| | tgcggtcccgccctcagcagtttctagagaaccatcagatgtttccagggt | |
| | gccccaaggacctgaaatgaccctgtgccttatttgaactaaccaatcagtt | |
| | cgcttctcgcttctgttcgcgcgcttctgctccccgagctcaataaaagagc | |
| | ccacaacccctcactcggcgcgccagtcctccgatagactgcgtcgccc | |
| | gggtacccgtgttctcaataaaccctcttgcagttgcatccgactcgtggtct | |
| | cgctgttccttgggagggtctcctctgagtgattgactgcccacctcgggg | |
| | gtctttcattctcgagcagcttggcgtaatcatggtcatagctgtttcctgtgt | |
| | gaaattgttatccgctcacaattccacacaacatacgagccggaagcataa | |
| | agtgtaaagcctggggtgcctaatgagtgagctaactcacattaattgcgtt | |
| | gcgctcactgcccgctttccagtcgggaaacctgtcgtgccagctgcatta | |
| | atgaatcggccaacgcgcggggagaggcggtttgcgtattgggcgctctt | |
| | ccgcttcctcgctcactgactcgctgcgctcggtcgttcggctgcggcgag | |
| | cggtatcagctcactcaaaggcggtaatacggttatccacagaatcaggg | |
| | gataacgcaggaaagaacatgtgagcaaaaggccagcaaaaggccag | |
| | gaaccgtaaaaaggccgcgttgctggcgtttttccataggctccgcccccc | |
| | tgacgagcatcacaaaaatcgacgctcaagtcagaggtggcgaaacccg | |
| | acaggactataaagataccaggcgtttccccctggaagctccctcgtgcg | |
| | ctctcctgttccgaccctgccgcttaccggatacctgtccgcctttctcccttc | |
| | gggaagcgtggcgctttctcatagctcacgctgtaggtatctcagttcggtg | |
| | taggtcgttcgctccaagctgggctgtgtgcacgaaccccccgttcagccc | |
| | gaccgctgcgccttatccggtaactatcgtcttgagtccaacccggtaaga | |
| | cacgacttatcgccactggcagcagccactggtaacaggattagcagagc | |
| | gaggtatgtaggcggtgctacagagttcttgaagtggtggcctaactacgg | |
| | ctacactagaagaacagtatttggtatctgcgctctgctgaagccagttacc | |
| | ttcggaaaaagagttggtagctcttgatccggcaaacaaaccaccgctggt | |
| | agcggtggtttttttgtttgcaagcagcagattacgcgcagaaaaaaaggat | |
| | ctcaagaagatcctttgatcttttctacggggtctgacgctcagtggaacga | |
| | aaactcacgttaagggattttggtcatgagattatcaaaaaggatcttcacct | |
| | agatccttttaaattaaaaatgaagttttaaatcaatctaaagtatatatgagta | |
| | aacttggtctgacagttaccaatgcttaatcagtgaggcacctatctcagcg | |
| | atctgtctatttcgttcatccatagttgcctgactccccgtcgtgtagataacta | |
| | cgatacgggagggcttaccatctggccccagtgctgcaatgataccgcga | |
| | gacccacgctcaccggctccagatttatcagcaataaaccagccagccgg | |
| | aagggccgagcgcagaagtggtcctgcaactttatccgcctccatccagt | |
| | ctattaattgttgccgggaagctagagtaagtagttcgccagttaatagtttg | |
| | cgcaacgttgttgccattgctacaggcatcgtggtgtcacgctcgtcgtttg | |
| | gtatggcttcattcagctccggttcccaacgatcaaggcgagttacatgatc | |
| | ccccatgttgtgcaaaaaagcggttagctccttcggtcctccgatcgttgtc | |
| | agaagtaagttggccgcagtgttatcactcatggttatggcagcactgcata | |
| | attctcttactgtcatgccatccgtaagatgcttttctgtgactggtgagtactc | |
| | aaccaagtcattctgagaatagtgtatgcggcgaccgagttgctcttgccc | |
| | ggcgtcaatacgggataataccgcgccacatagcagaactttaaaagtgc | |
| | tcatcattggaaaacgttcttcggggcgaaaactctcaaggatcttaccgct | |
| | gttgagatccagttcgatgtaacccactcgtgcacccaactgatcttcagca | |
| | tcttttactttcaccagcgtttctgggtgagcaaaaacaggaaggcaaaatg | |
| | ccgcaaaaaagggaataagggcgacacggaaatgttgaatactcatactc | |
| | ttcctttttcaatattattgaagcatttatcagggttattgtctcatgagcggata | |
| | catatttgaatgtatttagaaaaataaacaaataggggttccgcgcacatttc | |
| | cccgaaaagtgccacctgacgtctaagaaaccattattatcatgacattaac | |
| | ctataaaaataggcgtatcacgaggccattcgtctcgcgcgtttcggtgat | |
| | gacggtgaaaacctctgacacatgcagctcccggagacggtcacagctt | |
| | gtctgtaagcggatgccgggagcagacaagcccgtcagggcgcgtcag | |
| | cgggtgttggcgggtgtcggggctggcttaactatgcggcatcagagca | |
| | gattgtactgagagtgcaccatatgcggtgtgaaataccgcacagatgcgt | |
| | aaggagaaaataccgcatcaggcgccattcgccattcaggctgcgcaact | |
| | gttgggaagggcgatcggtgcgggcctcttcgctattacgccagctggcg | |
| | aaagggggatgtgctgcaaggcgattaagttgggtaacgccagggttttc | |
| | ccagtcacgacgttgtaaaacgacggccagtgaattagtact | |
| SEQ ID NO: | ¹¹LSVMGLRIL¹⁹ | α-chain transmembrane |
| 16 | | |
| SEQ ID NO: | ¹¹LLVIVLRIL¹⁹ | hydrophobic |
| 17 | | substitutions to the a- |
| | | chain transmembrane |
| SEQ ID NO: | SGSG | Linker Amino Acid |
| 18 | | Sequence |
| SEQ ID NO: | tctggaagcggc | DNA sequence encoding |
| 19 | | Linker Sequence |
| SEQ ID NO: | RAKR | Furin Cleavage Site |
| 20 | | |
| SEQ ID NO: | agagccaagaga | DNA sequence encoding |
| 21 | | Furin Cleavage Site |
| SEQ ID NO: | MWGVFLLYVSMKMGGTT | Exemplary TCRα Signal |
| 22 | | peptide |
| SEQ ID NO: | MKSLRVLLVILWLQLSWVWSQ | Exemplary TCRα Signal |
| 23 | | peptide |

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is asset forth in the following claims.

1. A vector comprising a nucleic acid sequence encoding a recombinant therapeutic T cell receptor (TCR) specific to a Human Papilloma virus serotype 16 (HPV16) E7 protein peptide-MHC (pMHC) complex and a microRNA (miRNA) cassette targeting the constant domain of the endogenous human TCR α and β chains, wherein the recombinant therapeutic TCR comprises a fully human constant region.

2. The vector of claim 1, wherein the miRNA cassette comprises a nucleic acid sequence that is at least 90% identical to any one of SEQ ID NO: 7-10.

3. The vector of claim 1, wherein the miRNA cassette comprises a nucleic acid sequence that is at least 90% identical any one of SEQ ID NO: 11-14.

4. The vector of claim 1, wherein the miRNA cassette comprises a nucleic acid sequence that is at least 90% identical to SEQ ID NO: 6.

5. The vector of claim 1, wherein the vector is an adenoviral vector, an adenovirus-associated vector, a DNA vector, a lentiviral vector, a plasmid, a retroviral vector, or an RNA vector.

6. The vector of claim 1, wherein the recombinant therapeutic TCR is specific to a peptide-MHC (pMHC) complex comprising the amino acid sequence of SEQ ID NO: 1.

7. The vector of claim 1, wherein the recombinant therapeutic TCR is specific to an HLA-A*02:01/YMLDLQPET peptide-MHC (pMHC) complex.

8. The vector of claim 1, wherein the recombinant therapeutic TCR comprises an α chain that comprises the amino acid sequence of SEQ ID NO: 2 or the amino acid sequence of SEQ ID NO: 4.

9. The vector of claim 1, wherein the recombinant therapeutic TCR comprises a β chain that comprises the amino acid sequence of SEQ ID NO: 3 or the amino acid sequence of SEQ ID NO: 5.

10. The vector of claim 1, wherein the recombinant therapeutic TCR comprises TCR α and TCR β chains that are codon optimized.

11. The vector of claim 1, wherein the miRNA cassette is present in a pMP71 retroviral vector between splice donor and splice acceptor sites downstream of the 5′LTR.

12. A vector substantially as described in FIG. 7.

13. A cell comprising the vector of claim 1.

14. The cell of claim 13, wherein, when the recombinant therapeutic T cell receptor (TCR) binds to pMHC, the cell produces at least interferon gamma (IFNγ).

15. A composition comprising a plurality of cells of claim 13.

16. A composition comprising a vector of claim 1.

17. A method for manufacturing a cell expressing a therapeutic T cell receptor (TCR), comprising a step of transducing a cell with the vector of claim 1.

18. A method for treating a HPV associated cancer comprising administering to a subject in need thereof the cell of claim 13.

19. The method of claim 18, wherein the HPV associated cancer is HPV16-associated cancer.

20. The method of claim 18, wherein the HPV associated cancer is an oropharyngeal cancer or a cervical cancer.


SEQUENCE LISTING (23 sequences)

SEQ ID NO: 1 - protein, 9 aa, Human papillomavirus type 16
YMLDLQPET

SEQ ID NO: 2 - protein, 109 aa, ARTIFICIAL
  POLYPEPTIDE
GQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIP

SEQ ID NO: 3 - protein, 116 aa, Artificial Sequence
  POLYPEPTIDE
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVL

SEQ ID NO: 4 - protein, 271 aa, Artificial Sequence
  POLYPEPTIDE
MKSLRVLLVILWLQLSWVWSQGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 5 - protein, 314 aa, Artificial Sequence
  POLYPEPTIDE
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 6 - DNA, 312 bp, Artificial Sequence
  miRNA cassette
cttatcctctggctgctggaggcttgctgaaggctgtatgctgtgaaagtttaggttcgtatctgttttggcctctgactgacagatacgaataaactttcacaggacacaaggcctgttactagcactcacatggaacaaatggccacacgcgccaagagaacaaagtggagtctttgttgcccacacccagcttccctggctctctgatggctcaaacacagcgagtacatgagacacgctgtgtttcagccatcggtgagcttgggaagcatctgcagcagagcctgcctggtggcccctgagagattt

SEQ ID NO: 7 - DNA, 149 bp, Artificial Sequence
  miRNA cassette
cttatcctctggctgctggaggcttgctgaaggctgtatgctgtgaaagtttaggttcgtatctgttttggcctctgactgacagatacgaataaactttcacaggacacaaggcctgttactagcactcacatggaacaaatggccac

SEQ ID NO: 8 - DNA, 21 bp, Artificial Sequence
  miRNA Cassette
tgaaagtttaggttcgtatct

SEQ ID NO: 9 - DNA, 19 bp, Artificial Sequence
  miRNA Cassette
gttttggcctctgactgac

SEQ ID NO: 10 - DNA, 19 bp, Artificial Sequence
  miRNA cassette
agatacgaataaactttca

SEQ ID NO: 11 - DNA, 157 bp, Artificial Sequence
  miRNA cassette
caagagaacaaagtggagtctttgttgcccacacccagcttccctggctctctgatggctcaaacacagcgagtacatgagacacgctgtgtttcagccatcggtgagcttgggaagcatctgcagcagagcctgcctggtggcccctgagagattt

SEQ ID NO: 12 - DNA, 22 bp, Artificial Sequence
  miRNA cassette
tctgatggctcaaacacagcga

SEQ ID NO: 13 - DNA, 11 bp, Artificial Sequence
  miRNA cassette
gtacatgagac

SEQ ID NO: 14 - DNA, 22 bp, Artificial Sequence
  miRNA cassette
acgctgtgtttcagccatcggt

SEQ ID NO: 15 - DNA, 6717 bp, Artificial Sequence
  expression vector
ctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagcacgcgccttatcctctggctgctggaggcttgctgaaggctgtatgctgtgaaagtttaggttcgtatctgttttggcctctgactgacagatacgaataaactttcacaggacacaaggcctgttactagcactcacatggaacaaatggccacacgcgccaagagaacaaagtggagtctttgttgcccacacccagcttccctggctctctgatggctcaaacacagcgagtacatgagacacgctgtgtttcagccatcggtgagcttgggaagcatctgcagcagagcctgcctggtggcccctgagagatttacgcgtattcatgcatgacaaagttaagtaatagtccctctctccaagctcacttacaggcggccgcgccaccatgggacctggattgctttgttgggccctgctgtgtctgcttggagctggacttgtggatgccggcgtgacacagtctcccacacacctgatcaagaccagaggccagcaagtgaccctgagatgtagccctaagagcggccacgacaccgtgtcttggtatcagcaggctcttggccagggacctcagttcatcttccagtactacgaggaagaggaacggcagcggggcaacttccctgatagattctctggccatcagttccccaactacagcagcgagctgaacgtgaacgctctgctgctgggcgatagcgccctgtatctgtgtgccagttctcttggttggagaggcggcagatacaacgagcagttctttggccctggcaccagactgaccgtgctggaagatctgaagaacgtgttcccacctgaggtggccgtgtttgagccttctgaggccgagatcagccacacacagaaagccacactcgtgtgcctggccaccggcttttatcccgatcacgtggaactgtcttggtgggtcaacggcaaagaggtgcacagcggcgttagcacagaccctcagcctctgaaagagcagcccgctctgaacgacagcagatactgtctgagcagcagactgagagtgtccgccaccttctggcagaaccccagaaaccacttcagatgccaggtgcagttctacggcctgagcgagaatgacgagtggacccaggatagagccaagcctgtgacacagattgtgtctgccgaagcctggggcagagccgattgtggctttacaagcgagtcttaccagcagggcgtgctgagcgccacaatcctgtatgagatcctgctgggcaaagccactctgtacgctgtgctggtttctgccctggtgctgatggctatggtcaagcggaaggatagcagaggcagagccaagagatctggaagcggcgccacaaacttctcactgctgaaacaggccggcgacgtggaagagaaccctggacctatgaagtccctgcgggtgctgctggttattctgtggctgcagctgagctgggtttggagccagggacagaacatcgaccagcctaccgagatgacagccaccgaaggcgccatcgtgcagatcaattgcacctaccagaccagcggcttcaacggcctgttctggtatcaacagcatgccggcgaggcccctaccttcctgagctataatgtgctggacggcctggaagaaaagggcagattcagcagcttcctgtccagaagcaagggctacagctacctgctgctgaaagaactccagatgaaggacagcgcctcctacctgtgtgcctccgtggatggaaacaacagactggccttcggcaagggcaaccaggtggtggtcatccccgacattcagaaccctgatcctgccgtgtaccagctgagagacagcaagagcagcgacaagagcgtgtgtctgttcaccgacttcgacagccagaccaacgtgtcccagagcaaggactccgacgtgtacatcaccgacaagacagtgctggacatgcggagcatggacttcaagagcaatagcgccgtggcctggtccaacaagagcgattttgcctgcgccaacgccttcaacaacagcatcatccctgaggacacattcttcccaagtcctgagagcagctgcgacgtgaagctggtggaaaagagcttcgagacagacaccaacctgaacttccagaacctgagcgtgatcggcttccggatcctgcttctgaaggtggccggcttcaatctgctgatgacactgagactgtggtccagctgaattcggatccaagcttaggcctgctcgctttcttgctgtcccatttctattaaaggttcctttgttccctaagtccaactactaaactgggggatattatgaagggccttgagcatctggattctgcctagcgctaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcattctcgagcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtact

SEQ ID NO: 16 - protein, 9 aa, Artificial Sequence
  polypeptide
LSVMGLRIL

SEQ ID NO: 17 - protein, 9 aa, Artificial Sequence
  polypeptide
LLVIVLRIL

SEQ ID NO: 18 - protein, 4 aa, Artificial Sequence
  polypeptide
SGSG

SEQ ID NO: 19 - DNA, 12 bp, Artificial Sequence
  pulynucleotide
tctggaagcggc

SEQ ID NO: 20 - protein, 4 aa, Artificial Sequence
  furin cleavage site
RAKR

SEQ ID NO: 21 - DNA, 12 bp, Artificial Sequence
  polynucleotide
agagccaagaga

SEQ ID NO: 22 - protein, 17 aa, Artificial Sequence
  polypeptide
MWGVFLLYVSMKMGGTT

SEQ ID NO: 23 - protein, 21 aa, Artificial Sequence
  polypeptide
MKSLRVLLVILWLQLSWVWSQ